SEQUENCE LISTING (7 sequences)

SEQ ID NO: 1 - DNA, 19 bp, synthetic construct
  source 1..19: mol_type = other DNA
tccctatcagtgatagaga

SEQ ID NO: 2 - DNA, 20 bp, synthetic construct
  source 1..20: mol_type = other DNA
ggccgagatgtctcgctccg

SEQ ID NO: 3 - DNA, 19 bp, synthetic construct
  source 1..19: mol_type = other DNA
gagaagatcccgttcatcc

SEQ ID NO: 4 - DNA, 20 bp, synthetic construct
  source 1..20: mol_type = other DNA
gctacccgcgcgagaattgc

SEQ ID NO: 5 - DNA, 18 bp, synthetic construct
  source 1..18: mol_type = other DNA
acccagactggtgcaaga

SEQ ID NO: 6 - DNA, 18 bp, synthetic construct
  source 1..18: mol_type = other DNA
gctcttctctgggctctg

SEQ ID NO: 7 - DNA, 19 bp, synthetic construct
  source 1..19: mol_type = other DNA
gtcaggaaagtccaacgcc

(19) United States
(12) Patent Application Publication
(10) Pub. No.: US 2026/0317766 A1
(43) Pub. Date: Oct. 15, 2026

(54) NOVEL METHOD TO MODULATE EXPRESSION OF AN ENDOGENOUS GENE IN A SOMATIC CELL

(71) Applicant: Bit Bio Limited, Cambridge (GB)

(72) Inventors: Mark Kotter, Cambridge (GB); Sejla Salic-Hainzl, Cambridge (GB); Emmanouil Metzakopian, Cambridge (GB); Nora Papai, Cambridge (GB)

(21) Appl. No.: 19/639,987

(22) Filed: Apr. 6, 2026

Related U.S. Application Data
(63) Continuation of application No. PCT/GB2024/052603, filed on Oct. 10, 2024.

(30) Foreign Application Priority Data
Oct. 10, 2023 (EP) 23202777.1
Oct. 4, 2024 (EP) 24300007.2

(51) Int. Cl. C12N 15/113; C07K 14/47; C12N 5/00; C12N 5/079; C12N 9/22; C12N 15/52

(52) U.S. Cl. CPC C12N 15/113; C07K 14/4702; C12N 5/0622; C12N 5/562; C12N 9/226; C12N 15/52; C12N 2310/20; C12N 2506/45; C12N 2510/00; C12N 2830/002; C12N 2830/34

(57) ABSTRACT

The invention relates to a method of modulating the expression of an endogenous gene in a somatic cell derived from an induced pluripotent stem cell (iPSC), comprising differentiating an engineered iPSC capable of stable or inducible expression of a CRISPR-related protein into a somatic cell prior to introduction of a guide RNA (gRNA) sequence. The invention also relates to the engineered iPSC, cells obtained by the method and uses thereof.

### SEQUENCE LISTING

The instant application contains a Sequence Listing in XML format which has been submitted via Patent Center and is hereby incorporated by reference in its entirety. The XML file, created on Apr. 6, 2026, is named BBIO-012USWOC1_SL.xml, and is 7,167 bytes in size.

### FIELD OF THE INVENTION

The present invention relates to methods of modulating the expression of an endogenous gene in a somatic cell derived from an induced pluripotent stem cell (iPSC) and iPSC cell lines used in such methods. In particular, the invention relates to differentiation methods where a guide RNA involved in modulating the target gene, is only introduced once the cell is no longer pluripotent.

### BACKGROUND

Stem cell research holds great promise for research of human development, regenerative medicine, disease modelling, drug discovery, and cell transplantation. Moreover, stem cell-derived cells enable studying physiological and pathological responses of human cell populations that are not easily accessible. This often entails the study of genes (and other forms of regulatory mechanisms encoded in non-protein-coding RNAs). Unfortunately, controllable transcription or expression of genetic information in human cells has been proven to be particularly difficult.

Moreover, for several key aspects of regenerative medicine, disease modelling, drug discovery and cell transplantation, manipulation and manufacture of mature human cell types from easily accessible sources is required. Controlling the expression of transgenes in human cells is the basis of biological research. However, this has proven to be difficult in human cells. Moreover, there is a real need for the in vitro derivation of many highly desirable human cell types in a quantity and quality suitable for drug discovery and regenerative medicine purposes. Because directed differentiation of stem cells into desired cell types is often challenging, other approaches have emerged, including direct reprogramming of cells into the desired cell types. In particular, forward programming as a method of directly converting pluripotent stem cells to mature cell types has been recognised as a powerful strategy for the derivation of human cells. This reprogramming involves the forced expression of key lineage transcription factors in order to convert the stem cell into a particular mature cell type.

Any refinements to the above methods must ensure that stable transcription of the genetic material contained within the inducible cassette, such as a transgene, is achieved which is resistant to silencing and other negative integration site-related influences. Silencing may be caused by multiple epigenetic mechanisms, including DNA methylation or histone modifications. For many applications, it is also desirable to control the transcription of inserted genetic material in a cell, such that an inducible cassette may be turned on as required and transcribed at particular levels, including high levels.

WO2018096343 describes a dual genomic safe harbour targeted system. It comprises a method for introducing an inducible cassette into a cell and permitting controllable transcription from within that inducible cassette using a system split across two genomic safe harbour sites.

The inventors have developed a method for enabling the stable reprogramming of pluripotent stem cells, whilst also optimising gene editing in somatic cells using the CRISPR/Cas system. This has benefits because the approach specifically reveals genetic perturbation phenotypes in mature cells.

### SUMMARY OF THE INVENTION

According to a first aspect of the invention, there is provided a method of modulating the expression of an endogenous gene in a somatic cell derived from an induced pluripotent stem cell (iPSC), comprising the steps of:
(i) providing an engineered iPSC capable of stable or inducible expression of a CRISPR enzyme, a catalytically-inactive CRISPR enzyme or a derivative thereof;
(ii) inducing the engineered iPSC to differentiate into the somatic cell; and
(iii) introducing a guide RNA (gRNA) sequence into the somatic cell obtained in step (ii), wherein said gRNA sequence is complementary to the endogenous gene,
   thereby modulating the expression of the endogenous gene in the somatic cell following differentiation.

According to a further aspect of the invention, there is provided a cell with a modified genome that comprises:
(i) an inserted genetic sequence encoding a transcriptional regulator protein at a first genomic safe harbour (GSH) site;
(ii) an inserted inducible cassette comprising a genetic sequence operably linked to an inducible promoter at a second GSH site, wherein said inducible promoter is regulated by the transcriptional regulator protein; and
(iii) an inserted genetic sequence encoding a CRISPR enzyme, a catalytically-inactive CRISPR enzyme or a derivative thereof operably linked to a promoter;
wherein the first and second GSH sites are different, and
wherein the genetic sequence encoding the CRISPR enzyme, catalytically-inactive CRISPR enzyme or derivative thereof is inserted at a site which is different to the first and second GSH sites.

According to a further aspect of the invention, there is provided an engineered iPSC comprising a genetic sequence encoding a CRISPR enzyme, a catalytically-inactive CRISPR enzyme or a derivative thereof operably linked to a promoter and optionally a reporter gene and a self-cleaving peptide, wherein said genetic sequence is inserted into a GSH site and wherein said GSH site comprises no other exogenous coding sequence.

According to a further aspect of the invention, there is provided a cell obtained by the method as defined herein.

According to a further aspect of the invention, there is provided the cell as defined herein, for use in therapy, in vitro diagnostics, or screening (such as drug screening, drug target identification, drug target validation or functional genomic screening).

### BRIEF DESCRIPTION OF THE FIGURES

FIG. 1: Cell line engineering strategy for Cas9 insertion. Knock-in of CAG driven rtTA into the ROSA26 locus, doxycycline-inducible NEUROG2 (NGN2) expression cassette into the AAVS1 locus and CAG driven Cas9-2A-mCherry cassette into the CLYBL locus.

FIG. 2: Flow cytometric analysis of the activity of Cas9 in engineered iPSCs demonstrated by the knockout of B2M. The figure shows unstained and untransduced control (left), stained and untransduced control (middle) and stained and transduced sample (right).

FIG. 3: Comparative Immunofluorescence Staining of Glutamatergic Neurons and Glutamatergic Neurons CRISPR-Ready. In the Upper Panel, the staining profile for MAP2 and TUBB3 is displayed, highlighting dendritic structures and neuronal differentiation respectively. The Lower Panel presents the staining characteristics for MAP2 and VGLUT2, indicating dendritic structures and glutamatergic neuron identification respectively.

FIG. 4: Principal Component Analysis Displaying Temporal Transcriptomic Differences between Glutamatergic Neurons and Glutamatergic Neurons CRISPR-Ready. This plot visualizes the transcriptional profiles of both cell lines at distinct timepoints: iPSCs stage, day 4, and day 18 post-cryopreservation. Circles denote the “Glutamatergic Neurons”, whereas triangles represent the “Glutamatergic Neurons CRISPR-ready”. Variations in positioning within the plot highlight the transcriptional differences and similarities between the two cell lines across the selected timepoints.

FIG. 5: Western Blot analysis of Cas9 expression in Glutamatergic Neurons CRISPR-Ready over time. The blot demonstrates the temporal dynamics of Cas9 protein levels at specified timepoints: Day −3, Day −1, Day 4 (if cells would be cryopreserved), and Day 6 (if cells would be cryopreserved). The observed decrease in Cas9 band intensity indicates reduced Cas9 expression in Glutamatergic Neurons CRISPR-Ready with prolonged culture times. Beta-actin is used as the loading control to ensure consistent protein loading across samples.

FIG. 6: Amplicon sequencing analysis of indel formation in the SOX11 gene. The figure shows percentage of indel formation within the targeted region of the SOX11 gene. Guide RNAs were delivered using two methods: RNAiMAX, a lipid-based method, and Lentivirus. Indel formation results are compared between guide deliveries on Day 1 and Day 3, with cells analyzed 5 days after the guide delivery. A non-targeting guide serves as the control, offering a comparative baseline for the efficiency of the targeted modifications.

FIG. 7: Immunofluorescence staining analysis of SOX11 expression following lipid-based Guide RNA delivery. Images of immunofluorescence staining for SOX11 after guide RNA delivery using the RNAiMAX lipid-based approach. SOX11 expression levels are compared between guide deliveries on Day 1 and Day 3, with cells analyzed at Day 11 of forward programming. DAPI staining provides visualization of the cell nuclei, serving as a reference for cellular localization. A non-targeting guide acts as the control, offering a comparative baseline for assessing SOX11 expression changes.

FIG. 8: Immunofluorescence staining analysis of SOX11 expression following lentiviral Guide RNA delivery. This figure showcases the outcomes of immunofluorescence staining for SOX11 following guide RNA introduction via Lentivirus. Differences in SOX11 expression are contrasted between guide deliveries on Day 1 and Day 3, with cells assessed at Day 11 of forward programming. DAPI is employed to stain cell nuclei, aiding in cell localization and density interpretation. A non-targeting guide is used as the control to provide a benchmark for evaluating SOX11 expression variations.

FIG. 9: UMAP Visualization from a pooled scCRISPR Screen demonstrating transcriptomic similarity among specific gene Knock-outs. Presented are four distinct UMAP plots, each characterizing the transcriptomic profiles of cells with knock-outs for AARS1, GARS1, HARS1, or CARS1. In each plot, cells with these specific knock-outs cluster together, emphasizing their transcriptomic congruence. All four genes are key players in the unfolded protein response and are associated with the neurological condition known as Charcot-Marie-Tooth disease. The evident clustering not only underscores the functional interplay of these genes in the disease but also validates the potential of these cells for robust CRISPR screening.

FIG. 10: Principal Component Analysis Displaying Temporal Transcriptomic Differences between Microglia and Microglia CRISPR-Ready. This plot visualizes the transcriptional profiles of both cell lines at distinct timepoints: iPSCs stage, day 10 (prior cryopreservation), and day 20 (10 days post-cryopreservation). Circles denote the “Microglia”, whereas triangles represent the “Microglia CRISPR-ready”. Variations in positioning within the plot highlight the transcriptional differences and similarities between the two cell lines across the selected timepoints.

FIG. 11: Cytokine secretion profile of Microglia and Microglia CRISPR-Ready. Cytokine production from Microglia and Cas9 expressing Microglia +/−LPS treatment shows comparable secretion profiles. The secretion profiles were assessed using the Proteome Profiler Human Cytokine Array kit (R&D Systems).

FIG. 12: Functionality of Cas9 in forward programmed Microglia. The figure shows knock-out efficiency in forward programmed Microglia at Day 9. Guide RNA targeting B2M was delivered by lentiviral transduction at D9 after cryopreservation. Cells were stained with B2M antibody and knock-out efficiency was assessed by flow cytometry (86.4%). A non-targeting guide serves as a control, offering a comparative baseline for the efficiency of the targeted modifications. Cells were stained for CD11b which is a marker of microglial cells.

FIG. 13: Functionality of Cas9 in forward programmed Microglia over time. Cells were transduced at different timepoints post revival (D1, D6, D9, D18). Guide RNA targeting B2M was delivered by lentiviral transduction. Cells were stained with B2M antibody and knock-out efficiency was assessed by flow cytometry 5 days post transduction (Knockout efficiencies at different timepoints of guide delivery; D1: 86.9%, D6: 84.8%, D9: 86.4%, D18: 78.4%). Cells were stained for CD11b which is a marker of microglial cells.

FIG. 14: Transcriptomic profile of Gene Knock-Outs from scCRISPR Screen. Heatmap illustrating genes identified as an activation signature through bulk RNA-seq of both +/−LPS treated cells (y-axis). Cosine similarity analysis employed non-targeting guides from both +/−LPS conditions. Knockouts in the LPS condition that result in cells exhibiting greater similarity to the non-targeting-no-LPS condition are plotted along the x-axis. Genes marked in grey are associated with the TLR4 pathway, responsible for microglial activation in response to LPS treatment.

FIG. 15: Cell line engineering strategy for dCas9-VPR insertion. Knock-in of CAG driven rtTA into the ROSA26 locus, doxycycline-inducible NEUROG2 (NGN2) expression cassette into the AAVS1 locus and CAG driven dCas9-VPR cassette into the CLYBL locus.

FIG. 16: Flow cytometric analysis of the activity of dCas9-VPR in engineered iPSCs demonstrated by the activation of CD274. The figure shows unstained and untransduced control (left), stained and non-targeting control (middle) and stained and sgRNA CD274 transduced sample (right).

FIG. 17: Flow cytometric analysis of the activity of dCas9-VPR in glutamatergic neurons demonstrated by the activation of CD274. Functionality of dCas9-VPR was demonstrated at three different timepoints (left: Day 1, middle: Day 3, right: Day 11). Upper panel shows transduction efficiency based on GFP signal. Lower panel confirms activation of CD274 compared to a non-targeting guide RNA at all three timepoints.

FIG. 18: Cell line engineering strategy for dCas9-ZIM3 insertion. Knock-in of CAG driven rtTA into the ROSA26 locus, doxycycline-inducible NEUROG2 (NGN2) expression cassette into the AAVS1 locus and CAG driven dCas9-ZIM3 cassette into the CLYBL locus.

FIG. 19: Immunofluorescence staining analysis of SOX11 expression following lentiviral Guide RNA delivery. This figure showcases the outcomes of immunofluorescence staining for SOX11 following guide RNA introduction via Lentivirus. Differences in SOX11 expression are contrasted between cell transduced with a SOX11 targeting guide RNA, non-targeting guide RNA (NTV) and untransduced cells (No VR). Two different virus dilutions of the SOX guide RNA were used 1:500 and 1:1000. DAPI is employed to stain cell nuclei, aiding in cell localization and knock down interpretation.

FIG. 20: Cell line engineering strategy for Cas9 insertion. Knock-in of CAG driven rtTA into the ROSA26 locus, doxycycline-inducible OLIG2-SOX10 expression cassette into the AAVS1 locus and CAG driven Cas9 cassette into the CLYBL locus.

FIG. 21: Flow cytometric analysis of Cas9 functionality in engineered iPSCs demonstrated by the B2M knockout efficiency. The figure shows two clones (P1D1-heterozygous and P1F6-homozygous) transduced with a non-targeting control (upper panel) and B2M targeting guide RNA (lower panel). Cells were stained with B2M antibody and high knockout efficiency is confirmed in both clones (P1D1: 84% B2M-negative cells; P1F6: 91% B2M-negative cells).

FIG. 22: Functionality of Cas9 in forward programmed glutamatergic neurons on Day 11. A guide RNA targeting SOX11 (sgRNA_SOX11) was delivered by both, lipid-based and lentiviral transduction into glutamatergic neurons. Cells were stained with SOX11 antibody five days post guide RNA delivery and knock-out efficiency was assessed by immunofluorescence staining. A non-targeting guide RNA (sgRNA_NT) as well as untransduced (No Virus) cells were used as a control for benchmarking purposes. Image analysis indicates a significant knock-out of SOX11 protein following introduction of the SOX11-specific gRNA.

### DETAILED DESCRIPTION OF THE INVENTION

The invention describes methods using an induced pluripotent stem cell (iPSC line) that has been engineered to express a CRISPR-related protein from a genomic safe harbour locus but wherein these cells are devoid of any guide RNA until they have been differentiated to a somatic cell. The iPSCs expressing the CRISPR-related protein are differentiated into specific somatic cells, such as neurons, using directed differentiation or forward programming methods. Post differentiation, and importantly after the loss of pluripotency, one or more guide RNAs are introduced to the somatic cells, allowing for targeted genome editing at this stage.

The core of this invention lies in the strategic combination of stable CRISPR-related protein expression in iPSCs, the absence of guide RNA at the pluripotent stage, and the introduction of the guide RNA post-differentiation, ensuring precise genome editing in non-pluripotent somatic cells. The incorporation of cryopreservation further enhances the applicability of this system, ensuring its relevance in large-scale applications.

As described herein, the methodology involves:
1. Engineering iPSCs to express a CRISPR-related protein, such as from a genomic safe harbour locus.
2. Optionally cryopreserving the CRISPR-related protein-expressing pre-differentiated cells for scalability and long-term use.
3. Initiating differentiation of these cells into specific somatic cells without the presence of any guide RNA.
4. Introducing guide RNA once cells have exited pluripotency for targeted genome editing.

Therefore, according to a first aspect of the invention, there is provided a method of modulating the expression of an endogenous gene in a somatic cell derived from an iPSC, comprising the steps of:
(i) providing an engineered iPSC capable of stable or inducible expression of a CRISPR enzyme, a catalytically-inactive CRISPR enzyme or a derivative thereof;
(ii) inducing the engineered iPSC to differentiate into the somatic cell; and
(iii) introducing a guide RNA (gRNA) sequence into the somatic cell obtained in step (ii), wherein said gRNA sequence is complementary to the endogenous gene,
   thereby modulating the expression of the endogenous gene in the somatic cell following differentiation.

In one embodiment, the engineered iPSC comprises an inserted genetic sequence encoding the CRISPR enzyme, catalytically-inactive CRISPR enzyme or a derivative thereof operably linked to a promoter.

A “promoter” is a nucleotide sequence which initiates and regulates transcription of a polynucleotide. It is intended that the term “promoter” or “control element” includes full-length promoter regions and functional (e.g., controls transcription or translation) segments of these regions. “Operably linked” refers to an arrangement of elements wherein the components so described are configured so as to perform their usual function. Thus, a given promoter operably linked to a genetic sequence is capable of effecting the expression of that sequence when the proper enzymes are present. The promoter need not be contiguous with the sequence, so long as it functions to direct the expression thereof. Thus, for example, intervening untranslated yet transcribed sequences can be present between the promoter sequence and the genetic sequence and the promoter sequence can still be considered “operably linked” to the genetic sequence. Thus, the term “operably linked” is intended to encompass any spacing or orientation of the promoter element and the genetic sequence in the inducible cassette which allows for initiation of transcription of the inducible cassette upon recognition of the promoter element by a transcription complex.

In one embodiment, the promoter is a constitutive promoter. Constitutive promoters ensure sustained and high level gene expression. Commonly used constitutive promoters include the human β-actin promoter (ACTB), cytomegalovirus (CMV), elongation factor-1α, (EF1α), phosphoglycerate kinase (PGK) and ubiquitinC (UbC). The CAG promoter is a strong synthetic RNA Polymerase II promoter frequently used to drive high levels of gene expression and was constructed from the following sequences: (C) the cytomegalovirus (CMV) early enhancer element, (A) the promoter, the first exon and the first intron of chicken beta-actin gene, and (G) the splice acceptor of the rabbit beta-globin gene. Further examples of suitable constitutive promoters include RNA Polymerase III promoters, such as the human U6 promoter, mouse U6 promoter or human H1 promoter. RNA Polymerase III promoters, such as the ones mentioned above, are typically used to express guide RNAs.

In alternative embodiment, the promoter is an inducible promoter. An “inducible promoter” is a nucleotide sequence where expression of a genetic sequence operably linked to the promoter is controlled by an analyte, co-factor, regulatory protein, etc.

In one embodiment, the inserted genetic sequence additionally comprises a reporter gene. Marker or reporter genes encode molecules that induce visually identifiable characteristics including fluorescent and luminescent proteins. Examples include the gene that encodes jellyfish green fluorescent protein (GFP), which causes cells that express it to glow green under blue/UV light, luciferase, which catalyses a reaction with luciferin to produce light, and the red fluorescent protein from the gene dsRed or derivatives thereof, such as mCherry. Such markers or reporter genes are useful, since the presence of the reporter protein confirms protein expression of the CRISPR-related protein, indicating successful insertion.

In one embodiment, the inserted genetic sequence comprises at least two coding regions, wherein the coding regions are linked to each other by nucleic acids that encode self-cleaving peptides so as to form a single open reading frame. As such, the inserted genetic sequence additionally comprises a self-cleaving peptide, such as between the CRISPR enzyme and the reporter gene. In one embodiment, the self-cleaving peptide is a viral 2A peptide. Self-cleaving peptides are found in members of the Picornaviridae virus family, including aphthoviruses such as foot-and-mouth disease virus (FMDV), equine rhinitis A virus (ERAV), Thosea asigna virus (TaV) and porcine teschovirus-1 (PTV-1). The 2A peptides derived from FMDV, ERAV, PTV-1, and TaV are sometimes referred to as “F2A”, “E2A”, “P2A”, and “T2A”, respectively. The 2A sequence is believed to mediate ‘ribosomal skipping’ between the proline and glycine, impairing normal peptide bond formation between the P and G without affecting downstream translation.

Engineered iPSCs

The engineered iPSC is capable of stable or inducible expression of a CRISPR enzyme, a catalytically-inactive CRISPR enzyme or a derivative thereof.

The CRISPR-related protein may be delivered by a vector, such as a viral vector. A “vector” is a nucleic acid molecule, such as a DNA molecule, which is used as a vehicle to artificially transfer exogenous genetic material into a cell. The vector is generally a nucleic acid sequence that consists of an insert (such as a sequence encoding the CRISPR-related protein) and a larger sequence that serves as the “backbone” of the vector. The vector may be in any suitable format, including plasmids, minicircle, linear DNA or a single-stranded AAV template. The vector comprises at least the sequence for encoding the CRISPR-related protein, together with the minimum sequences to enable insertion of the sequence. Optionally, the vectors also possess an origin of replication (ori) which permits amplification of the vector, for example in bacteria. Additionally or alternatively, the vector includes selectable markers such as antibiotic resistance genes, genes for coloured markers and/or suicide genes.

Vectors include but are not limited to plasmids, cosmids, viruses (bacteriophage, animal viruses and plant viruses) and artificial chromosomes (e.g., yeast artificial chromosomes or YACs).

In one embodiment, the vector(s) is a viral vector. The viral gene delivery system may be an RNA-based or DNA-based viral vector. Viral vectors include retroviral vectors (e.g., lentiviral vectors, such as those derived from HIV-1, HIV-2, SIV, BIV, FIV etc.), gammaretroviral vectors, adenoviral (Ad) vectors (including replication competent, replication deficient and gutless forms thereof), adeno-associated virus-derived (AAV) vectors, simian virus 40 (SV-40) vectors, bovine papilloma virus vectors, Epstein-Barr virus vectors, herpes virus vectors, vaccinia virus vectors, Harvey murine sarcoma virus vectors, murine mammary tumour virus vectors, Rous sarcoma virus vectors and Sendai virus vectors. In a further embodiment, the viral vector(s) is selected from: a lentiviral vector, an adeno-associated virus vector or a Sendai virus vector. In a yet further embodiment, the viral vector(s) is a lentiviral vector.

Lentiviral vectors are well known in the art. Lentiviral vectors are complex retroviruses capable of integrating randomly into the host cell genome, which, in addition to the common retroviral genes gag, pol, and env, contain other genes with regulatory or structural function (e.g., accessory genes Vif, Nef, Vpu, Vpr). Lentiviral vectors have the advantage of being able to infect non-dividing cells and can be used for both in vivo and ex vivo gene transfer and expression of nucleic acid sequences. For example, a recombinant lentiviral vector is capable of infecting a non-dividing cell wherein a suitable host cell is transfected with two or more vectors carrying the packaging functions, namely gag, pol and env, as well as rev and tat.

In another embodiment, the vector(s) is a plasmid. The plasmid(s) may be episomal. Episomal vectors are able to introduce large fragments of DNA into a cell but are maintained extra-chromosomally, replicated once per cell cycle, partitioned to daughter cells efficiently, and elicit substantially no immune response. Alternatively, an Epstein-Barr virus (EBV)-based episomal vector, a yeast-based vector, an adenovirus-based vector, a simian virus 40 (SV40)-based episomal vector, or a bovine papilloma virus (BPV)-based vector may be used.

In another embodiment, the vector(s) is a transposon (i.e. involving a transposon plasmid). A transposon delivery system is comprised of two plasmids, one encoding the transposase and one encoding the genetic sequence to be delivered. The transposase protein may mediate random integration of the transcripts encoded in the transposon plasmid into the genome. In one embodiment, the transposon system is selected from a PiggyBac or Sleeping Beauty transposon system. The transposon plasmid encodes a payload/genetic sequence flanked by two ITRs (internal terminal repeats).

In one embodiment, the transposase and transposon plasmids are delivered by nucleofection or lipofection into the cells. The number of integration events, and therefore the number of copies of payload per cell can be in part controlled by adjusting the total and relative amounts of transposase and transposon plasmid DNA.

In one embodiment, the engineered iPSC comprises a genetic sequence encoding the CRISPR enzyme, catalytically-inactive CRISPR enzyme or derivative thereof at a target site, such as a genomic safe harbour (GSH) site. Targeted integration may be achieved using one or more of the methods described herein.

The iPSC may be engineered to comprise other genetic material. Such material includes reporter genes, suicide genes and selectable markers. Genes for marker or reporter molecules are discussed herein, such as genes that induce visually identifiable characteristics including fluorescent and luminescent proteins. Selectable markers may further include resistance genes to antibiotics or other drugs. Non-inducible expression of molecular tools may also be desirable, including optogenetic tools, nuclear receptor fusion proteins, such as tamoxifen-inducible systems ERT, and designer receptors exclusively activated by designer drugs. Furthermore, sequences that code signalling factors that alter the function of the same cell or of neighbouring or even distant cells in an organism, including hormones, autocrine or paracrine factors may be co-expressed.

Additionally, the additional genetic material may include sequences coding for non-coding RNA, as discussed herein. Examples of such genetic material include gRNA sequences and genes for miRNA, which may function as a genetic switch.

### CRISPR Enzyme

It will be understood that references herein to “CRISPR-related protein” refer to a CRISPR enzyme, a catalytically-inactive CRISPR enzyme or a derivative thereof. The “CRISPR enzyme” is a major protein component of a clustered regularly interspaced short palindromic repeats (CRISPR)-CRISPR-associated protein (Cas) system and forms a complex with a guide RNA (gRNA), thereby forming a CRISPR-Cas system.

Three types of CRISPR mechanisms have been identified, of which type II is the most studied. The CRISPR/Cas9 system (type II) utilises the Cas9 nuclease to make a double-stranded break in DNA at a site determined by a short guide RNA (also referred to herein as simply, guide RNA). The CRISPR/Cas system is a prokaryotic immune system that confers resistance to foreign genetic elements. CRISPR are segments of prokaryotic DNA containing short repetitions of base sequences. Each repetition is followed by short segments of “protospacer DNA” from previous exposures to foreign genetic elements. CRISPR spacers recognize and cut the exogenous genetic elements using RNA interference. The CRISPR immune response occurs through two steps: CRISPR-RNA (crRNA) biogenesis and crRNA-guided interference. CrRNA molecules are composed of a variable sequence transcribed from the protospacer DNA and a CRISPR repeat. Each crRNA molecule then hybridizes with a second RNA, known as the trans-activating CRISPR RNA (tracrRNA) and together these two eventually form a complex with the nuclease Cas9. The protospacer DNA encoded section of the crRNA directs Cas9 to cleave complementary target DNA sequences, if they are adjacent to short sequences known as protospacer adjacent motifs (PAMs). This natural system has been engineered and exploited to introduce DSB breaks in specific sites in genomic DNA, amongst many other applications. In particular, the CRISPR type II system from Streptococcus pyogenes may be used. At its simplest, the CRISPR/Cas9 system comprises two components that are delivered to the cell to provide genome editing: the Cas9 nuclease itself and a gRNA. The gRNA is a fusion of a customised, site-specific crRNA (directed to the target sequence) and a standardised tracrRNA.

In one embodiment, the CRISPR enzyme is Cas9. In another embodiment, the CRISPR enzyme is Cas12a/Cpf1. CRISPR enzymes are known in the art, such as those described in Wang et al. Genome Biol. 19, 62 (2018). Other CRISPR enzymes have been utilized in human cells and are known to the skilled person.

Once a double-strand DNA break (DSB) has been made, a donor template with homology to the targeted locus can be supplied. Distinct cellular repair mechanisms can be exploited to repair the DSB and to introduce the desired sequence, and these are non-homologous end joining repair (NHEJ), which is more prone to error; and homologous recombination repair. The DSB may be repaired by the homology-directed repair (HDR) pathway allowing for precise insertions to be made.

In one embodiment, the endogenous gene contains an insertion or deletion after introduction of the gRNA sequence, thus disrupting the coding sequence of the endogenous gene (CRISPR knockout). In a further embodiment, the endogenous gene is fully or partially deleted after introduction of the gRNA sequence.

Derivatives of the CRISPR/Cas system are also possible. For example, mutant forms of Cas9 are available, such as Cas9D10A, with only nickase activity. This means it cleaves only one DNA strand, and does not activate NHEJ. Instead, when provided with a homologous repair template, DNA repairs are conducted via the high-fidelity HDR pathway only. Cas9D10A may be used in paired Cas9 complexes designed to generate adjacent DNA nicks in conjunction with two sgRNAs complementary to the adjacent area on opposite strands of the target site.

### Catalytically-Inactive CRISPR Enzyme

In one embodiment, the engineered iPSC is capable of stable or inducible expression of a catalytically-inactive CRISPR enzyme or derivative thereof. Catalytically-inactive CRISPR enzymes may also be referred to as ‘dead’. The programmable nuclease may be rendered catalytically inactive through point mutations in the endonuclease domain(s), such as the RuvC and HNH domains in Cas9. Examples of such point mutations in the endonuclease domains of catalytically inactive Cas9 (also known as dCas9) are D10A and H840A which result in its deactivation. However, as will be readily appreciated such mutations do not affect the ability of the programmable nuclease to bind gRNA and to the targeted gene as such binding is affected by other domains.

In one embodiment, the catalytically-inactive CRISPR enzyme is catalytically inactive Cas9 (i.e. dead Cas9 or dCas9). In another embodiment, the catalytically-inactive CRISPR enzyme is catalytically inactive Cas12a (i.e., dCas12a). In further embodiments, the catalytically-inactive CRISPR enzyme (e.g., dCas9 or dCas12a) is a derivative that comprises point mutations in the RuvCI and HNH nuclease domains. In a particular embodiment, the catalytically-inactive CRISPR enzyme is dCas9 and comprises the point mutations D10A and H840A compared to the wild-type sequence of Cas9.

The methods presented herein may utilise CRISPR activation (CRISPRa) which uses modified versions of CRISPR effectors which do not have endonuclease activity but comprise added transcriptional activators on the catalytically-inactive CRISPR enzyme (e.g. dCas9 and dCas12a fused to transcriptional activators) and/or the gRNAs. The transcriptional activators fused to the CRISPRa components or which bind thereto (e.g., through the presence of aptamer sequences) therefore increase expression of genes of interest following targeting to the gene by the gRNA.

Therefore, in one embodiment, the expression of the endogenous gene is activated (CRISPR activation). In this embodiment, one or more transcription activator proteins may be fused to the catalytically-inactive CRISPR enzyme. In a further embodiment, the catalytically-inactive CRISPR enzyme is dCas9 and is fused to the transcription activator proteins VP64, p65 and Rta (i.e. the catalytically inactive programmable nuclease is dCas9-VPR). In another embodiment, the gRNA sequence is engineered to comprise a transcription activation domain which is capable of recruiting one or more transcription activator proteins to upregulate expression of the endogenous gene.

The methods presented herein may utilise CRISPR interference (CRISPRi) which uses modified versions of CRISPR effectors which do not have endonuclease activity but comprise added transcriptional repressors on the catalytically-inactive CRISPR enzyme (e.g. dCas9 and dCas12a fused to transcriptional repressors) and/or the gRNAs.

Therefore, in one embodiment, the expression of the endogenous gene is repressed (CRISPR interference). In this embodiment, one or more transcription repressor proteins is fused to the catalytically-inactive CRISPR enzyme. In one embodiment, transcriptional repressors are chosen from the KRAB repressors, in particular the KRAB domain of KOX1 or ZIM3. According to this embodiment, the catalytically-inactive CRISPR enzyme may be dCas9 fused to the KRAB domain of KOX1 or ZIM3 (i.e. the catalytically-inactive CRISPR enzyme may be dCas9-KOX1 or dCas9-ZIM3). In another embodiment, the gRNA sequence is engineered to comprise a transcription repression domain which is capable of recruiting one or more transcription repressor proteins to downregulate expression of the endogenous gene.

In one embodiment, the gRNA sequence is complementary to a coding exon of the endogenous gene. Depending on the CRISPR-related protein targeted by the gRNA, affecting the coding exon of an endogenous gene will result in modulation of the expression of said endogenous gene.

In one embodiment, the gRNA sequence is complementary to a transcription start site (TSS) for the endogenous gene. Depending on the CRISPR-related protein targeted by the gRNA, affecting the TSS of an endogenous gene will result in modulation of the expression of said endogenous gene.

Guide RNA (gRNA) Sequences

The “gRNA” refers to an RNA capable of specifically targeting a CRISPR complex, that is, a gRNA-CRISPR enzyme complex, with respect to a target gene or nucleic acid. The gRNA is specific RNA for a target sequence (i.e. the endogenous gene), which may bind to the CRISPR enzyme, and guide the CRISPR enzyme to the target gene or nucleic acid.

Thus, the gRNA sequence targets the CRISPR enzyme, a catalytically-inactive CRISPR enzyme or a derivative thereof, to the endogenous gene to modulate its expression.

Methods of delivering the RNA into the cell are known in the art. The gRNA may be a synthetic RNA or it may be incorporated in a plasmid or incorporated in a lentiviral donor plasmid. The gRNA may be delivered by a vector, such as a viral vector, as described hereinbefore. In one embodiment, the gRNA is introduced by viral transduction. In another embodiment, the gRNA is introduced using lipid-based transfection or electroporation of a gRNA expressing vector. In another embodiment, the gRNA is introduced using lipid-based transfection or electroporation of a synthetic gRNA.

In one embodiment, the gRNA is introduced within about 6 days of differentiation. In a further embodiment, the gRNA is introduced within 1-3 days from differentiation.

In one embodiment, two or more gRNA sequences are introduced. In a further embodiment, each of the gRNA sequences are complementary to alternative or different endogenous genes, or are complementary to more than one alternative sequences of a TSS for the endogenous gene.

According to embodiments where multiple (e.g., two or more), or a pool of gRNA sequences are introduced, said multiple gRNA sequences may be separated by cleavable sequences. Cleavable sequences are sequences that are recognised by an entity capable of specifically cutting DNA, and include restriction sites, which are the target sequences for restriction enzymes or sequences for recognition by other DNA cleaving entities, such as nucleases, recombinases, ribozymes or artificial constructs. At least one cleavable sequence may be included. These cleavable sequences may be at any suitable point, such that a selected portion of the sequence, or all of the sequence, can be selectively removed. The cleavable sites may thus flank the part/all of the sequence may be subsequently removed. Such cleavable sequences may be recognised by nucleases, recombinases, ribozymes or artificial constructs (e.g., a bacterial DNA endonuclease, such as Csy4). In one embodiment, the multiple gRNA sequences are provided in an array comprising a promoter, two or more gRNA sequences and two or more cleavable sequences. In a particular embodiment, the multiple gRNA sequences are provided in an array comprising a constitutive promoter (e.g. the hU6 promoter), two gRNA sequences and two cleavable sequences. In a further embodiment, each guide RNA sequence may be driven by a separate promoter, e.g. a first gRNA driven by the human U6 promoter, a second gRNA driven by the mouse U6 promoter, a third gRNA driven by the bovine U6 promoter, a fourth gRNA driven by the H1 promoter and so on.

In another embodiment, the two or more gRNA sequences are introduced separately, e.g., by plasmid transfection, electroporation, lentiviral infection or AAV infection. In one embodiment, a lentiviral vector is used that harbours a single gRNA expression cassette. In a further embodiment, a gRNA library is introduced into the lentiviral vector and provided to the target cell for pooled or arrayed CRISPR screening.

In one embodiment, the one or more gRNA sequences are operably linked to a constitutive promoter. In one embodiment, the promoter is an RNA Polymerase III-driven promoter, such as human U6, mouse U6, bovine U6 or the human H1 promoter.

### Genomic Safe Harbour (GSH) Sites & Insertions Therein

The methods described herein involve targeting safe harbour sites in the genome of an iPSC. Insertions specifically within genomic safe harbour sites are preferred over random genome integration, since this is expected to be a safer modification of the genome, and is less likely to lead to unwanted side effects such as silencing of natural gene expression or causing mutations that lead to cancerous cell types.

A genomic safe harbour (GSH) site is a locus within the genome wherein a gene or other genetic material may be inserted without any deleterious effects on the cell or on the inserted genetic material. Most beneficial is a GSH site in which expression of the inserted gene sequence is not perturbed by any read-through expression from neighbouring genes and expression of the inducible cassette minimizes interference with the endogenous transcription programme. More formal criteria have been proposed that assist in the determination of whether a particular locus is a GSH site in future (Papapetrou et al. (2011) Nature Biotechnology, 29 (1): 73-8, doi: 10.1038/nbt.1717). These criteria include a site that is (i) 50 kb or more from the 5′ end of any gene, (ii) 300 kb or more from any gene related to cancer, (iii) 300 kb or more from any microRNA (miRNA), (iv) located outside a transcription unit and (v) located outside ultra-conserved regions (UCR). It may not be necessary to satisfy all of these proposed criteria, since GSH sites already identified do not fulfil all of the criteria. It is thought that a suitable GSH site will satisfy at least 2, 3, 4 or all of these criteria.

Further sites may be identified by looking for sites where viruses naturally integrate without disrupting native gene expression. They may also be defined as sites that cannot be silenced and thus warrant stable transgene expression, such as described in WO2021152086A1 and which are hereby incorporated by reference.

Any suitable GSH site may be used in the method of the invention, on the basis that the site allows insertion of genetic material without deleterious effects to the cell and permits transcription of the inserted genetic material. Those skilled in the art may use this simplified criterion to identify a suitable GSH site, and/or the more formal criteria set out above.

For the human genome, several GSHs have been identified, and these include the AAVS1 locus, the hROSA26 locus and the CLYBL gene. The CCR5 gene has also been mooted as a possible GSH, and further investigation may identify one or more of these as GSHs in the human genome. Additional GSHs have recently been discovered and validated by Aznauryan et al. (Cell Rep Methods, 2022; 2 (1): 100154), https://doi.org/10.1016/j.crmeth.2021.100154) which are hereby incorporated by reference.

The adeno-associated virus integration site 1 locus (AAVS1) is located within the protein phosphatase 1, regulatory subunit 12C (PPP1R12C) gene on human chromosome 19, which is expressed uniformly and ubiquitously in human tissues. This site serves as a specific integration locus for AAV serotype 2, and thus was identified as a possible GSH. AAVS1 has been shown to be a favourable environment for transcription, since it comprises an open chromatin structure and native chromosomal insulators that enable resistance of the inducible cassettes against silencing. There are no known adverse effects on the cell resulting from disruption of the PPP1R12C gene. Moreover, an inducible cassette inserted into this site remains transcriptionally active in many diverse cell types. AAVS1 is thus considered to be a GSH site and has been widely utilized for targeted transgenesis in the human genome.

The hROSA26 site has been identified on the basis of sequence analogy with a GSH site from mice (ROSA26—reverse oriented splice acceptor site #26). Although the orthologue site has been identified in humans, this site is not commonly used for inducible cassette insertion. The present inventors have developed a targeting system specifically for the hROSA26 site and thus were able to insert genetic material into this locus. The hROSA26 locus is on chromosome 3 (3p25.3), and can be found within the Ensembl database (GenBank: CR624523). The integration site lies within the open reading frame (ORF) of the THUMPD3 long non-coding RNA (reverse strand). Since the hROSA26 site has an endogenous promoter, the inserted genetic material may take advantage of that endogenous promoter, or alternatively may be inserted operably linked to an exogenous promoter.

Intron 2 of the Citrate Lyase Beta-like (CLYBL) gene, on the long arm of Chromosome 13, was identified as a suitable GSH site since it is one of the identified integration hot-spots of the phage derived phiC31 integrase. Studies have demonstrated that randomly inserted inducible cassettes into this locus are stable and expressed. It has been shown that insertion of inducible cassettes at this GSH site do not perturb local gene expression (Cerbibi et al, 2015, PLOS One, DOI: 10.1371). CLYBL thus provides a GSH site which may be suitable for use in the present invention.

CCR5, which is located on chromosome 3 (position 3p21.31) is a gene which codes for HIV-1 major co-receptor. Interest in the use of this site as a GSH arises from the null mutation in this gene that appears to have no adverse effects, but predisposes to HIV-1 infection resistance. Zinc-finger nucleases that target the third exon have been developed, thus allowing for insertion of genetic material at this locus. Given that the natural function of CCR5 has yet to be elucidated, the site remains a putative GSH site which may have utility for the present invention.

GSHs in other organisms have been identified and include ROSA26, HPRT and Hipp11 (H11) loci in mice. Mammalian genomes may include GSH sites based upon pseudo attP sites. For such sites, hiC31 integrase, the Streptomyces phage-derived recombinase, has been developed as a non-viral insertion tool, because it has the ability to integrate an inducible cassette-containing plasmid carrying an attB site into pseudo attP sites.

GSHs are also present in the genomes of plants, and modification of plant cells can form part of the present invention. GSH sites have been identified in the genomes of rice (Cantos et al. (2014) Front. Plant Sci., 5 (302), doi: http://dx.doi.org/10.3389/fpls.2014.00302).

Thus, in one embodiment the GSH sites are selected from the group consisting of: the hROSA26 locus, the AAVS1 locus, the CLYBL gene and the CCR5 gene, such as wherein the first genomic safe harbour site is the hROSA26 locus and the second genomic safe harbour site is the AAVS1 locus. In one embodiment the first, second and third GSH sites are selected from any three of: the hROSA26 locus, the AAVS1 locus, the CLYBL gene and/or the CCR5 gene, such as wherein the first genomic safe harbour site is the hROSA26 locus, the second genomic safe harbour site is the AAVS1 locus and the third genomic safe harbour site is the CLYBL gene. In another embodiment, the first and second GSH sites are selected from any two of: the hROSA26 locus, the AAVS1 locus, the CLYBL gene and/or the CCR5 gene, such as wherein the first genomic safe harbour site is the hROSA26 locus and the second genomic safe harbour site is the AAVS1 locus or the CLYBL gene.

In the methods of the invention, insertions occur at different GSH sites, thus at least two GSH sites are required. It will be understood that the reference to “different GSH sites” refers to GSH sites located at different locations in the genome.

In one embodiment, the genetic sequence encoding the transcriptional regulator protein is inserted at the first GSH site on both chromosomes of the cell. In one embodiment, the inducible cassette is inserted at the second GSH site on both chromosomes of the cell. In one embodiment, the genetic sequence encoding the CRISPR enzyme, catalytically-inactive CRISPR enzyme or derivative thereof is inserted at the third GSH site on both chromosomes of the cell.

In one embodiment, the engineered iPSC comprises additional genetic material inserted at the first, second and/or third GSH site. Such genetic material may be selected from one or more of the following: a reporter gene; a suicide gene; and a selectable marker.

### Site-Specific Delivery & Targeting

Preferably, the insertions into the GSH sites are specifically within the sequence of the GSH as described previously. Any suitable technique for insertion of a polynucleotide into a specific sequence may be used, and several are described in the art. Suitable techniques include any method which introduces a break at the desired location and permits recombination of a donor vector into the gap. Thus, a crucial first step for targeted site-specific genomic modification is the creation of a double-strand DNA break (DSB) at the genomic locus to be modified. Distinct cellular repair mechanisms can be exploited to repair the DSB and to introduce the desired sequence, and these are non-homologous end joining repair (NHEJ), which is more prone to error; and homologous recombination repair (HR) mediated by a donor DNA template, that can be used to insert inducible cassettes. In some embodiments, the insertions into the GSH sites are performed using the techniques disclosed in WO 2018/096343, which are hereby incorporated by reference. Thus, any method of making specific, targeted double strand breaks in the genome in order to insert a gene/inducible cassette may be used in the method of the invention. It may be preferred that the method for inserting the gene/inducible cassette utilises any one or more of ZFNs, TALENs and/or CRISPR/Cas9 systems or any derivative thereof.

Zinc finger nucleases are artificial enzymes which are generated by fusion of a zinc-finger DNA-binding domain to the nuclease domain of the restriction enzyme FokI. The latter has a non-specific cleavage domain which must dimerise in order to cleave DNA. This means that two ZFN monomers are required to allow dimerisation of the FokI domains and cleavage of the DNA. The ZFN DNA binding domain comprises a tandem array of Cys₂His₂ zinc fingers, each of which recognises three contiguous nucleotides in the target sequence, and may be designed to target any genomic sequence of interest. The two binding sites are separated by 5-7 bp to allow optimal dimerisation of the FokI domains. The catalytically capable enzyme is thus able to cleave DNA at a specific site. Target specificity is increased by ensuring that two proximal DNA-binding events must occur to achieve a double-strand break.

Transcription activator-like effector nucleases, or TALENs, are dimeric transcription factor/nucleases. They are made by fusing a TAL effector DNA-binding domain to a DNA cleavage domain (a deoxyribonuclease). Transcription activator-like effectors (TALEs) can be engineered to bind practically any desired DNA sequence, so when combined with a nuclease, DNA can be cut at specific locations. TAL effectors are proteins that are secreted by Xanthomonas bacteria, the DNA binding domain of which contains a repeated highly conserved 33-34 amino acid sequence with divergent 12th and 13th amino acids. These two positions are highly variable and show a strong correlation with specific nucleotide recognition.

This straightforward relationship between amino acid sequence and DNA recognition has allowed for the engineering of specific DNA-binding domains by selecting a combination of repeat segments containing appropriate residues at the two variable positions. TALENs are thus built from arrays of 33 to 35 amino acid modules, each of which targets a single nucleotide. By selecting the array of the modules, almost any sequence may be targeted.

Once the DSB has been made by any appropriate means, the gene/inducible cassette for insertion may be supplied in any suitable fashion as described below. The gene/inducible cassette and associated genetic material form the donor DNA for repair of the DNA at the DSB and are inserted using standard cellular repair machinery/pathways. How the break is initiated will alter which pathway is used to repair the damage, as noted above.

In some embodiments the targeted insertion is obtained in the absence of a DNA double-strand break. For example, this may be achieved using a viral vector, such as adeno-associated virus (AAV), for targeting. In this instance, the vector is modified to contain the transgene, alongside with a suitable selection marker (PuroR, BlaR, HygroR, ZeoR or eGFP, mCherry, DsRed) flanked by suitable homology arms. Infection of the target cells may produce a genetically engineered cell in which the target locus (e.g. GSH site) contains the transgene.

### Dual Safe-Harbour

It is highly desirable to provide in vitro methods of producing selected cell types in a quantity and quality suitable for drug discovery and regenerative medicine purposes. Directed differentiation of stem cells into desired cell types is often challenging, therefore other approaches have emerged, including direct reprogramming of cells. In particular, forward programming as a method of directly converting pluripotent stem cells, including iPSCs, to mature cell types has been recognised as a powerful strategy for the derivation of human cells. This reprogramming involves the forced expression of key lineage transcription factors (referred to herein as “lineage-specific transcription factors”) in order to convert the stem cell into a particular somatic cell type. However, robust, controlled expression of transgenes in pluripotent stem cells is generally challenging due to silencing mechanisms operating in the cells (e.g. as described in Pfaff et al. (2013) Stem Cells 31:488-499). Consistent, high efficiency of expression is critical during reprogramming to avoid proliferating pluripotent stem cells remaining and contaminating the resultant composition.

In one embodiment, the engineered iPSC additionally comprises:
(a) an inserted genetic sequence encoding a transcriptional regulator protein at a first GSH site;
(b) an inserted inducible cassette comprising a genetic sequence operably linked to an inducible promoter at a second GSH site, wherein said inducible promoter is regulated by the transcriptional regulator protein,
wherein the first and second GSH sites are different.

### Transcriptional Regulator Proteins & Inducible Transcription

A transcriptional regulator protein is a protein that binds to DNA, preferably sequence-specifically to a DNA site located in or near a promoter, and either facilitates the binding of the transcription machinery to the promoter, and thus transcription of the DNA sequence (e.g., a transcriptional activator) or blocks this process (e.g., a transcriptional repressor). Such entities are also known as transcription factors.

The DNA sequence that a transcriptional regulator protein binds to is called a transcription factor-binding site or response element, and these are found in or near the promoter of the regulated DNA sequence.

Transcriptional activator proteins bind to a response element and promote gene expression. Such proteins are preferred in the methods of the present invention for controlling inducible cassette expression.

Transcriptional repressor proteins bind to a response element and prevent gene expression. Transcriptional regulator proteins may be activated or deactivated by a number of mechanisms including binding of a substance, interaction with other transcription factors (e.g., homo- or hetero-dimerization) or coregulatory proteins, phosphorylation, methylation and/or by light. The transcriptional repressor may be controlled by activation or deactivation.

If the transcriptional regulator protein is a transcriptional activator protein, it is preferred that the transcriptional activator protein requires activation. This activation may be through any suitable means, but it is preferred that the transcriptional regulator protein is activated through the addition to the cell of an exogenous substance. The supply of an exogenous substance to the cell can be controlled, and thus the activation of the transcriptional regulator protein can be controlled. Alternatively, an exogenous substance can be supplied in order to deactivate a transcriptional regulator protein, and then withdrawn in order to activate the transcriptional regulator protein. Thus, in one embodiment the activity of the transcriptional regulator protein is controlled by an exogenous substance.

If the transcriptional regulator protein is a transcriptional repressor protein, it is preferred that the transcriptional repressor protein requires deactivation. Thus, a substance is supplied to prevent the transcriptional repressor protein repressing transcription, and thus transcription is permitted.

Any suitable transcriptional regulator protein may be used, preferably one that is activatable or deactivatable. It is preferred that an exogenous substance may be supplied to control the transcriptional regulator protein. Such transcriptional regulator proteins are also called inducible transcriptional regulator proteins.

Tetracycline-Controlled Transcriptional Activation is a method of inducible gene expression where transcription is reversibly turned on or off in the presence of the antibiotic tetracycline or one of its derivatives (e.g., doxycycline (which is more stable) or minocycline). In this system, the transcriptional activator protein is reverse tetracycline trans-activator (rtTa, which may also be referred to as a tetracycline-responsive transcriptional activator protein) or a derivative thereof. The rtTA protein is able to bind to DNA at specific tet operator (TetO) sequences. Several repeats of such TetO sequences are placed upstream of a minimal promoter (such as the CMV promoter), which together form a tetracycline response element (TRE). There are two forms of this system, depending on whether the addition of tetracycline or a derivative activates (Tet-On) or deactivates (Tet-Off) the rtTA protein.

In a Tet-Off system, tetracycline or a derivative thereof binds rtTA and deactivates the rtTA, rendering it incapable of binding to TRE sequences, thereby preventing transcription of TRE-controlled genes. This system was first described in Bujard, et al (1992). Proc. Natl. Acad. Sci. U.S.A. 89 (12): 5547-51.

The Tet-On system is composed of two components; (1) the constitutively expressed reverse tetracycline trans-activator (rtTa) protein and the rtTa sensitive inducible promoter (Tet Responsive Element, TRE). rtTA binds to the TRE-containing promoter in the presence of tetracycline or its more stable derivatives, including doxycycline (dox), resulting in activation of rtTa, inducing expression of TRE-controlled genes. The use of this may be preferred in the method of the invention.

Thus, the transcriptional regulator protein may be rtTa which can be activated or deactivated by the antibiotic tetracycline or one of its derivatives, which are supplied exogenously. If the transcriptional regulator protein is rtTA, then the inducible promoter inserted into the second GSH site includes the tetracycline response element (TRE). The exogenously supplied substance is the antibiotic tetracycline or one of its derivatives.

Variants and modified rtTa proteins may be used in the methods of the invention, these include Tet-On Advanced transactivator (also known as rtTA2S-M2) and Tet-On 3G (also known as rtTA-V16, derived from rtTA2S-S2).

The tetracycline response element (TRE) generally consists of 7 repeats of the 19 bp bacterial TetO sequence separated by spacer sequences, together with a minimal promoter. Variants and modifications of the TRE sequence are possible, since the minimal promoter can be any suitable promoter. Preferably the minimal promoter shows no or minimal expression levels in the absence of rtTa binding. The inducible promoter inserted into the second GSH site may thus comprise a TRE.

A modified system based upon tetracycline control is the T-REx™ System (Thermofisher Scientific), in which the transcriptional regulator protein is a transcriptional repressor protein, TetR. The components of this system include (i) an inducible promoter comprising a strong human cytomegalovirus immediate-early (CMV) promoter and two tetracycline operator 2 (TetO2) sites, and a Tet repressor (TetR). The TetO2 sequences consist of 2 copies of the 19 bp sequence, 5′-TCCCTATCAGTGATAGAGA-3′ (SEQ ID NO: 1) separated by a 2 bp spacer. In the absence of tetracycline, the Tet repressor forms a homodimer that binds with extremely high affinity to each TetO2 sequence in the inducible promoter, and prevents transcription from the promoter. Once added, tetracycline binds with high affinity to each Tet repressor homodimer rendering it unable to bind to the Tet operator. The Tet repressor: tetracycline complex then dissociates from the Tet operator and allows induction of expression. In this instance, the transcriptional regulator protein is TetR and the inducible promoter comprises two TetO2 sites. The exogenously supplied substance is tetracycline or a derivative thereof.

The cumate switch is another method of inducible gene expression where transcription is reversibly turned on or off in the presence of the cumate. This system is available in both activator and repressor configurations, where the presence of cumate leads to the repression of transcription or activation of transcription, respectively. In the repressor configuration, regulation is mediated by the binding of the repressor (CymR) to the operator site (CuO), placed downstream of a constitutive promoter. Addition of cumate, a small molecule, relieves the repression and allows transcription to proceed. In the activator configuration, a chimeric transactivator (cTA) protein, formed by the fusion of CymR with the activation domain of VP16, is able to activate transcription when bound to the CuO operator site, placed upstream of the constitutive promoter. Cumate addition abrogates DNA binding and therefore transactivation by cTA, stopping transcription.

Other inducible expression systems are known and can be used in the method of the invention. These include the Complete Control Inducible system from Agilent Technologies. This is based upon the insect hormone ecdysone or its analogue ponasterone A (ponA) which can activate transcription in mammalian cells which are transfected with both the gene for the Drosophila melongaster ecdysone receptor (EcR) and an inducible promoter comprising a binding site for the ecdysone receptor. The EcR is a member of the retinoid-X-receptor (RXR) family of nuclear receptors. In humans, EcR forms a heterodimer with RXR that binds to the ecdysone-responsive element (EcRE). In the absence of PonA, transcription is repressed by the heterodimer.

Thus, the transcriptional regulator protein can be a repressor protein, such as an ecdysone receptor or a derivative thereof. Examples of the latter include the VgEcR synthetic receptor from Agilent technologies which is a fusion of EcR, the DNA binding domain of the glutocorticoid receptor and the transcriptional activation domain of Herpes Simplex Virus VP16. The inducible promoter comprises the EcRE sequence or modified versions thereof together with a minimal promoter. Modified versions include the E/GRE recognition sequence of Agilent Technologies, in which mutations to the sequence have been made. The E/GRE recognition sequence comprises inverted half-site recognition elements for the retinoid-X-receptor (RXR) and GR binding domains. In all permutations, the exogenously supplied substance is ponasterone A, which removes the repressive effect of EcR or derivatives thereof on the inducible promoter, and allows transcription to take place.

Alternatively, inducible systems may be based on the synthetic steroid mifepristone as the exogenously supplied substance. In this scenario, a hybrid transcriptional regulator protein is inserted, which is based upon a DNA binding domain from the yeast GAL4 protein, a truncated ligand binding domain (LBD) from the human progesterone receptor and an activation domain (AD) from the human NF-κB. This hybrid transcriptional regulator protein is available from Thermofisher Scientific under the trade name Gene Switch™. Mifepristone activates the hybrid protein, and permits transcription from the inducible promoter which comprises GAL4 upstream activating sequences (UAS) and the adenovirus E1b TATA box. This system is described in Wang, Y. et al (1994) Proc. Natl. Acad. Sci. USA 91, 8180-8184.

The transcriptional regulator protein can thus be any suitable regulator protein, either an activator or repressor protein. Suitable transcriptional activator proteins are the reverse tetracycline trans-activator (rtTa) or the Gene Switch™ hybrid transcriptional regulator protein. Suitable repressor proteins include the Tet-Off version of rtTA, TetR or EcR. The transcriptional regulator proteins may be modified or derivatised as required.

Thus, in one embodiment the transcriptional regulator protein is selected from any one of: a reverse tetracycline trans-activator (rtTa) proteins, a tetracycline repressor (TetR), VgEcR synthetic receptor, a cumate repressor (CymR) or a hybrid transcriptional regulator protein comprising a DNA binding domain from the yeast GAL4 protein, a truncated ligand binding domain from the human progesterone receptor and an activation domain from the human NF-κB. In a particular embodiment the transcriptional regulator protein is rtTA and its activity is controlled by tetracycline of a derivative thereof, such as doxycycline. According to this particular embodiment, the inducible promoter may include a tetracycline-responsive element (TRE).

In one embodiment, the transcription of sequences inserted into the first GSH site is under the control of one or more constitutive promoters and the transcription of sequences inserted into the second GSH site is under the control of an inducible promoter. In another embodiment, the transcription of sequences inserted into the first and third GSH sites is under the control of one or more constitutive promoters and the transcription of sequences inserted into the second GSH site is under the control of an inducible promoter.

The inducible promoter can comprise elements which are suitable for binding or interacting with the transcriptional regulator protein. The interaction of the transcriptional regulator protein with the inducible promoter is preferably controlled by the exogenously supplied substance.

The exogenously supplied substance can be any suitable substance that binds to or interacts with the transcriptional regulator protein. Suitable substances include tetracycline, tetracycline derivatives (e.g., doxycycline), ponasterone A and mifepristone.

Thus, the insertion of the gene encoding a transcriptional regulator protein into the first GSH site provides the control mechanism for the expression of the inducible cassette which is operably linked to the inducible promoter and inserted into a second, different, GSH site.

It is preferred that the gene encoding the transcriptional regulator protein is operably linked to a constitutive promoter. Alternatively, the first GSH site can be selected such that it already has a constitutive promoter that can also drive expression of the transcriptional regulator protein gene and any associated genetic material.

Further, the transcriptional regulator, plus any additional genetic material may be provided together with cleavable sequences. Such sequences are sequences that are recognised by an entity capable of specifically cutting DNA, and include restriction sites, which are the target sequences for restriction enzymes or sequences for recognition by other DNA cleaving entities, such as nucleases, recombinases, ribozymes or artificial constructs. At least one cleavable sequence may be included, but preferably two or more are present. These cleavable sequences may be at any suitable point in the insertion, such that a selected portion of the insertion, or all of the insertion, can be selectively removed from the GSH site. The method can thus extend to removal and/or replacement of the insertion or a portion thereof from the GSH. The cleavable sites may thus flank the part/all of the insertion that it may be desired to remove. The transcriptional regulator and/or the further genetic material may be removed using this method. A portion of the insertion to be removed may be any part up to 99% of the insertion—i.e. 1-99%, 90%, 80%, 70%, 60%, 50%, 40%, 30%, 20%, 10% or less than 10%.

It may be preferred that the portion of the insertion flanked by the cleavable sites includes the constitutive promoter. Alternatively, the constitutive promoter is not included in the portion flanked by the cleavable sequences.

A preferred cleavable sequence is the IoxP site for Cre recombinase as it allows direct replacement of the removed insertion. Alternatively or additionally, the cleavable sequence is the rox site for Dre recombinase.

It is preferred that the insertion at the first GSH site occurs at both loci in the genome, thus each allele is modified by insertion. This permits greater expression from the gene encoding the transcriptional regulator and any associated genetic material.

### Inducible Cassettes

An inducible cassette includes a desired genetic sequence, preferably a DNA sequence that is to be transferred into a cell. In methods of the invention, the introduction of an inducible cassette into the genome has the potential to change the phenotype of that cell through activation of an endogenous gene.

In particular, the inducible cassette comprises a genetic sequence encoding a transgene for one or more master regulators. A “master regulator” is an expressed gene that influences the lineage of the cell expressing it. It may be that a network of master regulators is required for the lineage of a cell to be determined. As used herein, a master regulator gene that is expressed at the inception of a developmental lineage or cell type, participates in the specification of that lineage by regulating multiple downstream genes either directly or through a cascade of gene expression changes. If the master regulator is expressed it has the ability to re-specify the fate of cells destined to form other lineages. Examples of master regulators include transcription factors, transcriptional regulators, cytokine receptors or signalling molecules and the like.

In a further embodiment, the inducible cassette comprises a genetic sequence encoding a transcription factor. References to “transcription factor” as used herein, refer to proteins that are involved in gene regulation in both prokaryotic and eukaryotic organisms. In one embodiment, transcription factors can have a positive effect on gene expression and, thus, may be referred to as an “activator” or a “transcriptional activation factor”. In another embodiment, a transcription factor can negatively affect gene expression and, thus, may be referred to as “repressors” or a “transcription repression factor”. Activators and repressors are generally used terms and their functions may be discerned by those skilled in the art.

In a further embodiment, the inducible cassette comprises a genetic sequence encoding a lineage-specific transcription factor. References herein to “lineage-specific transcription factor” refer to proteins specifically or necessarily involved in the determination of the identity of a particular cell type, i.e. a lineage. The specific identity of lineage-specific transcription factor to be encoded will be appreciated to vary depending on the desired lineage of the somatic cell.

In one embodiment, transcription of the genetic sequence in the second GSH site (e.g. a lineage-specific transcription factor) results in the forward programming of the engineered iPSC into the somatic cell.

The second GSH site can be any suitable GSH site. It may be preferred that the second GSH site is not associated with an endogenous promoter, such that the expression of the inserted inducible cassette is solely under control of the transcriptional regulator protein.

### Differentiation Methods

According to the methods described herein, the engineered iPSC is induced to differentiate into a somatic cell.

In one embodiment, the method of inducing differentiation comprises forward programming. Forward programming comprises inducing the expression of one or more lineage-specific transcription factors. The generation of somatic cells using forward programming may be referred to as “cellular reprogramming”, “forward reprogramming”, “direct programming” or “direct differentiation”, i.e. the (pluripotent) stem cell is differentiated into a somatic cell. Furthermore, “cellular reprogramming” may be used as generic terminology referring to the use of transcription factors to differentiate a source cell into target somatic cells.

In one embodiment, forward programming comprises increasing the expression of one or more lineage-specific transcription factors in an iPSC and culturing the cell under conditions suitable for forward programming the cell into a somatic cell.

Forward programming may also be achieved by increasing the expression of the one or more endogenous lineage-specific transcription factor genes in an iPSC, for example using CRISPRa. Thus, in some embodiments the expression of the one or more endogenous lineage-specific factor genes is increased by the targeting of transcription activator proteins to one or more TSS of the endogenous lineage-specific transcription factor gene(s) by the one or more gRNA sequences which are complementary to said TSSs, e.g. through binding of at least one of the transcription activator proteins to an aptamer sequence in the gRNA, or through targeting a catalytically-inactive CRISPR enzyme to the TSS, e.g. because at least one of the transcription activator proteins are fused to the catalytically-inactive CRISPR enzyme. Therefore, in one embodiment, the gRNA sequence is complementary to a TSS for the endogenous lineage-specific transcription factor gene.

In one embodiment, the method of inducing differentiation comprises directed differentiation. Directed differentiation comprises culturing the iPSC under conditions required to induce differentiation into the somatic cell.

References herein to “culturing” include the addition of cells to media comprising growth factors and/or essential nutrients. It will be appreciated that such culture conditions may be adapted according to the cells or cell population to be generated.

In one embodiment, the engineered iPSC is frozen and subsequently thawed prior to differentiation. A distinguishing feature of the invention is the cryopreservation of the pre-differentiated cells harbouring a CRISPR-related protein. In one embodiment, differentiation is initiated and pre-differentiated cells, i.e. cells that have exited pluripotency, are frozen and subsequently thawed prior to differentiation. Cryopreservation is critical for practical application and scalability in manufacturing processes, as product line development necessitates the use of cryopreserved cells.

### Cells & Cell Compositions

The methods described herein involve differentiation of an iPSC into a somatic cell. iPSCs (induced pluripotent stem cells) are cells that have been reprogrammed to an embryonic stem cell-like state by being forced to express genes and factors important for maintaining the defining properties of embryonic stem cells. In 2006, it was shown that overexpression of four specific transcription factors could convert adult cells into pluripotent stem cells. Oct-3/4 and certain members of the Sox gene family have been identified as potentially crucial transcriptional regulators involved in the induction process. Additional genes including certain members of the Klf family, the Myc family, Nanog, and Lin28, may increase the induction efficiency. Examples of the genes which may be used as reprogramming factors to generate iPSCs include Oct3/4, Sox2, Sox1, Sox3, Sox15, Sox17, Klf4, Klf2, c-Myc, N-Myc, L-Myc, Nanog, Lin28, Fbx15, ERas, ECAT15-2, Tcl1, beta-catenin, Lin28b, SaII4, Esrrb, Tbx3 and Glis1, GATA3, GATA6 and these reprogramming factors may be used singly, or in combination of two or more kinds thereof. In particular, the reprogramming factors may comprise at least the Yamanaka factors, i.e., Oct3/4, Sox2, Klf4 and c-Myc. References herein to “pluripotent” refer to cells which have the potential to differentiate into all types/lineages of cell found in an organism. Multipotent stem cells are able to differentiate into a smaller number of cell types than pluripotent cells, such as only those of closely related cell lineages. Oligopotent stem cells can differentiate into only a few cell types, such as lymphoid or myeloid stem cells. Unipotent cells can produce only one cell type and are thus-lineage-specific but have the ability to self-renew which distinguishes them from non-stem cells (e.g., progenitor cells, which cannot self-renew). iPSCs are of particular interest to the present invention. Thus, it is preferred that cells used in the methods of the invention do not require destruction of an embryo, such as a human embryo. In some embodiments, the cells are not derived from human or animal embryos, i.e., the invention does not extend to any methods which involve the destruction of human or animal embryos.

In one embodiment, the iPSC is from a mammal. In a further embodiment, the mammal is a human. Thus, in a particular embodiment the iPSC is from a human and is thus a human stem cell. In an alternative embodiment, the iPSC is from a marsupial, non-human primate, camelid or livestock animal cell. Livestock animals include, for example, pigs, cows, horses, buffalo, bison, goats, sheep, deer, reindeer, donkeys, bantengs, yaks, chickens, ducks and turkeys.

In a further alternative embodiment, the mammal is a mouse, optionally such that the iPSC is a mouse stem cell. Methods of preparing induced pluripotent stem cells from mouse are known in the art. Induction of iPSCs typically requires the expression of or exposure to at least one member from Sox family and at least one member from Oct family. Sox and Oct are thought to be central to the transcriptional regulatory hierarchy that specifies ES cell identity. For example, Sox may be Sox-1, Sox-2, Sox-3, Sox-15, or Sox-18; Oct may be Oct-4. Additional factors may increase the reprogramming efficiency, like Nanog, Lin28, Klf4, or c-Myc; specific sets of reprogramming factors may be a set comprising Sox-2, Oct-4, Nanog and, optionally, Lin-28; or comprising Sox-2, Oct4, Klf and, optionally, c-Myc. In one method, the iPSC may be generated by transfecting cells with transcription factors Oct4, Sox2, c-Myc and Klf4 using viral transduction.

In one embodiment, the iPSC is derived from somatic or germ cells of a patient, such as a patient to be treated or subject in need of treatment. Such use of autologous cells would remove the need for matching cells to a recipient. Alternatively, commercially available iPSCs may be used, such as those available from WICELL (WiCell Research Institute, Inc, Wisconsin, US). Alternatively, the cells may be a tissue-specific stem cell which may also be autologous or donated.

The term “somatic cell” as used herein includes any type (i.e., lineage) of cell that makes up the body of an organism, excluding germ cells and undifferentiated stem cells. Methods of the invention may involve differentiating the iPSC into early or late somatic cells. Somatic cells may therefore include, for example and without limitation, neurons, glia cells, white blood cells (e.g., leucocytes), liver cells (e.g., hepatocytes), muscle cells (e.g., myocytes) or fibroblasts. In one embodiment, the somatic cell may be an adult cell or a cell derived from an adult which displays one or more detectable characteristics of an adult or non-embryonic cell.

References herein to a somatic cell differentiated from an iPSC refer to cells which comprise the phenotype and/or characteristics of a somatic cell (e.g. the surface phenotype and/or functional characteristics associated with a particular lineage).

The method may generate cells (i.e. differentiated cells) exhibiting at least one characteristic of a target somatic cell. One or more characteristics may be used to select for the somatic generated by methods of the invention.

Characteristics include but are not limited to the detection or quantitation of expressed cell markers, enzymatic activity, and the characterization of morphological features and intercellular signalling. The biological function of a target somatic cell may also be evaluated, for example using functional assays.

The method may comprise assaying the differentiated cells obtained by the method described herein and determining a set of transcribed genes; comparing the set of transcribed genes of the differentiated cells to one or more reference sets of transcribed genes from one or more reference somatic cells; and identifying a match between the differentiated cells and a reference somatic cell.

In one embodiment, the method comprises the step of identifying differentiated cells as a type of somatic cell by assaying morphological features of the differentiated cells and matching the morphological features to a reference tissue or cell's morphological features.

In one embodiment, the method comprises the step of identifying differentiated cells as a type of somatic cell by assaying protein marker expression of the differentiated cells and matching the protein marker expression to a reference somatic cell protein marker expression.

In one embodiment, the method comprises the step of identifying differentiated cells as a type of somatic cell by assaying a function and matching the function to a function of a reference somatic cell.

In one embodiment, the differentiated cells obtained by the methods of the invention express the target somatic cell phenotype. This phenotype may be defined through expression (+) or non-expression (−) of one of more markers. The differentiated cells may also be negative for markers of pluripotency (i.e. the differentiated cells are non-pluripotent).

Alternatively, certain differentiated cells may be sorted from other differentiated cells and from cells on the basis of their expression of a lineage-specific cell surface antigen. Yet another means is by assessing expression at the RNA level, e.g., by RT-qPCR methods or by single cell RNA sequencing without any sorting or pre-selection step. Such techniques are known in the art.

In one embodiment, the somatic cell is selected from: a neuronal cell (such as a GABAergic neurons, a sensory neuron, a glutamatergic neuron or a motor neuron), a glial cell (such as a microglial cell, an oligodendrocyte (or oligodendrocyte-like) or an astrocyte) or a muscle cell (such as a skeletal myocyte). In a further embodiment, the somatic cell is a neuronal cell, such as a glutamatergic neuron, or a glial cell, such as a microglial cell.

In one embodiment, the iPSC may be differentiated into a neuron, such as a glutamatergic neuron. Neurons may be defined by their morphology and/or function (e.g. neurotransmitter, polarity and signal direction). In a further embodiment, the neuron is a glutamatergic neuron. Such neurons may be characterised by the expression of the glutamate transporter genes VGLUT1 and VGLUT2.

Neurons may be generated through induction of one or more neuron-specific transcription factors genes. Examples of such genes include the endogenous NEUROG2 (neurogenin 2 or NGN2) gene and the NEUROD1 (neuronal differentiation 1) gene. Thus, in one embodiment, the method comprises inducing the engineered iPSC to differentiate into a neuron (such as a glutamatergic neuron) through induction of the NGN2 gene. Said induction may be through directed differentiation methods or forward programming (e.g. wherein the inserted inducible cassette in the second GSH site comprises a genetic sequence encoding NGN2).

In one embodiment, the iPSC may be differentiated into a glial cell, such as a microglial cell.

In some aspects of the invention, there is provided a cell obtained by the methods described herein. In a particular aspect, there is provided a somatic cell obtained by the methods described herein. Thus, in certain aspects of the invention there is provided a cell (e.g. a somatic cell) obtained by the methods of forward programming an iPSC comprising targeted insertion into three different GSH sites as described herein.

Therefore, in a further aspect of the invention, there is provided a cell with a modified genome that comprises:
(i) an inserted genetic sequence encoding a transcriptional regulator protein at a first GSH site;
(ii) an inserted inducible cassette comprising a genetic sequence operably linked to an inducible promoter at a second GSH site, wherein said inducible promoter is regulated by the transcriptional regulator protein; and
(iii) an inserted genetic sequence encoding a CRISPR enzyme, a catalytically-inactive CRISPR enzyme or a derivative thereof operably linked to a promoter;
wherein the first and second GSH sites are different, and
wherein the genetic sequence encoding the CRISPR enzyme, catalytically-inactive CRISPR enzyme or derivative thereof is inserted at a site which is different to the first and second GSH sites.

In one embodiment, the inserted genetic sequence encoding the CRISPR enzyme, catalytically-inactive CRISPR enzyme or derivative thereof is inserted at a third GSH site, wherein the third GSH site is different from the first and second GSH sites.

In one embodiment, the cell stably or inducibly expresses the CRISPR enzyme, catalytically-inactive CRISPR enzyme or derivative thereof.

As described herein, in a preferred embodiment the cell is an iPSC, in particular a human iPSC. In this embodiment, the cell does not comprise a gRNA sequence. The cells of the invention do not harbour guide RNA at the pluripotent stage.

In an alternative embodiment, the cell is a somatic cell differentiated from an iPSC, in particular a human somatic cell. In this embodiment, the cell additionally comprises an inserted genetic sequence encoding a gRNA sequence operably linked to a constitutive promoter.

In a further aspect of the invention, there is provided an engineered iPSC comprising a genetic sequence encoding a CRISPR enzyme, a catalytically-inactive CRISPR enzyme or a derivative thereof operably linked to a promoter and optionally a reporter gene and a self-cleaving peptide, wherein said genetic sequence is inserted into a GSH site and wherein said GSH site comprises no other exogenous coding sequence.

In one embodiment, the promoter is constitutive or inducible.

In one embodiment, the cell or the cell obtained by the methods described herein is provided in a pharmaceutical composition.

Pharmaceutical compositions may include cells as described herein in combination with one or more pharmaceutically or physiologically acceptable carrier, diluents, or excipients. Such compositions may include buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminium hydroxide); and preservatives. Cryopreservation solutions which may be used in the pharmaceutical compositions of the invention include, for example, DMSO.

For purposes of manufacture, distribution, and use, the cells described herein may be supplied in the form of a cell culture or suspension in an isotonic excipient or culture medium, optionally frozen to facilitate transportation or storage.

It will be readily understood that the methods described hereinbefore, or any/all steps thereof, may be performed in vivo, ex vivo or in vitro. In a particular embodiment, the methods described hereinbefore, or any/all steps thereof, are performed in vitro, such as ex vivo.

### Uses, Therapeutic Uses & Methods of Treatment

The cells produced according to any of the methods described herein have applications in basic and medical research, diagnostic and therapeutic methods. The cells may be used in vitro to study cellular development, provide test systems for new drugs, enable screening methods to be developed, scrutinise therapeutic regimens, provide diagnostic tests and the like. Inclusion of the CRISPR-related protein enables the generation of single gene knockouts, inhibitions or activations (e.g. using CRISPRi or CRISPRa approaches, respectively), and scale to high throughput pooled or arrayed CRISPR screens for applications including functional genomics, disease model generation, drug target identification and fundamental human biology research. Approaches may also combined such as for validation purposes, for example CRISPR knock out or inhibition screens can be validated using CRISPR activations or vice versa, wherein reciprocal results will be observed in activations compared to inhibitions/knock outs thereby increasing confidence in any identified target. These uses form part of the present invention. Alternatively, the cells may be transplanted into a human or animal patient for diagnostic or therapeutic purposes. The use of the cells in therapy is also included in the present invention.

Therefore, according to one aspect of the invention, there is provided the cell as defined herein for use in therapy.

According to another aspect of the invention, the cell defined herein is for use in the treatment of cancer, a neurological disorder, an inflammatory disease, an autoimmune disease and/or a chronic infectious disease.

According to another aspect of the invention, the cell defined herein is for use in in vitro diagnostics.

According to another aspect of the invention, the cell defined herein is for use in drug screening, drug target identification or drug target validation.

According to another aspect of the invention, the cell defined herein is for use in functional genomic screening. In particular, the cells defined herein find use in CRISPR screening and, for example, for use in identifying genotype-phenotype relationships in somatic cells derived from iPSCs.

Unless defined otherwise, all technical and scientific terms used herein have the meaning commonly understood by a person skilled in the art to which this invention belongs. As used herein, the term “about” when used herein includes up to and including 10% greater and up to and including 10% lower than the value specified, suitably up to and including 5% greater and up to and including 5% lower than the value specified, especially the value specified. The term “between” as used herein includes the values of the specified boundaries.

It will be understood that all embodiments described herein may be applied to all aspects of the invention and vice versa, and such combinations would be readily apparent from the description provided herein and to those skilled in the art.

Other features and advantages of the present invention will be apparent from the description provided herein. It should be understood, however, that the description and the specific examples while indicating preferred embodiments of the invention are given by way of illustration only, since various changes and modifications will become apparent to those skilled in the art.

The invention will now be described using the following, non-limiting examples:

### EXAMPLES

### Example 1: Gene Engineering Strategy for Cas9 Expressing Glutamatergic Neurons

### Methods

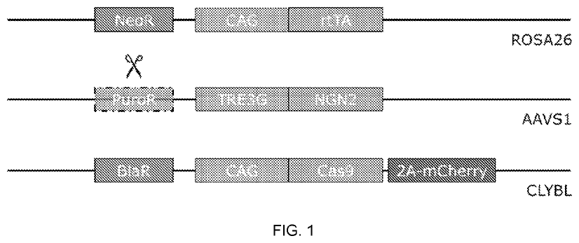

The aim of this experiment was to create an iPSC line that serves two purposes: (i) it can be forward programmed by the overexpression of transcription factors (e.g. NEUROG2 (NGN2) for Glutamatergic Neurons) and (ii) it expresses the Cas9 protein in a constitutive manner to enable gene editing in forward programmed/differentiated cells. Thereto, three transgenes were integrated into genome safe-harbour sites (GSH) by genome engineering (see FIG. 1). Firstly, a CAG promoter-driven reverse Tet transactivator (rtTA) cassette for doxycycline-controlled transactivation of a second recombinant transgene was integrated into the ROSA26 locus. Secondly, a transcription factor (e.g. NGN2) under the control of doxycycline-responsive promoter was integrated into the AAVS1 locus. Lastly, the CAG promoter-driven full-length Cas9 protein was integrated into the CLYBL locus, upstream of a P2A ribosomal-skipping sequence and a fluorescent protein for verification of integration.

### Experimental Outline

An iPSC line was cultured at all times. Editing of the safe harbour loci was performed subsequentially. All three loci were edited using CRISPR/Cas9 technology. A sgRNA which introduces a DNA double-strand break in close proximity to the chosen insertion site was selected. Cells were nucleofected with two plasmids—a plasmid expressing Cas9 and the corresponding sgRNA and a plasmid containing the insert sequence flanked by homology arms. The PAM sequence was mutated to prevent the re-cutting of Cas9.

Cells harbouring the correct integration were selected (e.g. using antibiotic resistance markers with the respective antibiotic). Once selection has reached a sufficient efficiency, cells were dissociated as single cells. To obtain monoclonal lines, cells were seeded at a low density to allow outgrowth of colonies originating from a single cell.

Engineered lines were submitted to two rounds of genotyping by PCR. The first round of genotyping was performed on crude lysate. Once clones with the correct integration were identified, cells were expanded, and genomic DNA was isolated for a second round of genotyping. PCRs for correct integration of 5′ and 3′ homology arms as well as the insert sequence were conducted. In addition, we performed PCRs to confirm if clones are heterozygous or homozygous for the intended integration.

### Results

The final iPSC line has been engineered to incorporate the dual safe harbour system, allowing transcription factor overexpression. It contains transcription factor(s) essential for forward programming into Glutamatergic neurons. Additionally, this line constitutively expresses the Cas9 protein, facilitating further genomic manipulations.

### Example 2: Benchmarking of Cas9 Expression and Activity at iPSC Stage

### Methods

To measure the activity of Cas9 at iPSC stage, a guide RNA targeting the B2 microglobulin (B2M) gene (GGCCGAGATGTCTCGCTCCG, SEQ ID NO: 2), which is expressed on all nucleated cells, was cloned into a lentiviral vector. The used vector encodes a puromycin-resistance cassette for the selection of cells with successful genomic integration. In addition, the vector contains GFP in order to estimate the multiplicity of infection. The lentiviral vector was used to transduce iPSCs harbouring an rtTA transactivator, constitutive full-length Cas9 and the transcription factor NGN2 necessary for forward programming under the control of a Tet-responsive promoter.

### Experimental Outline

Prior to lentiviral transduction, iPSCs were expanded on standard tissue culture plates. Cells were transduced with the lentiviral vector targeting B2M. Following transduction, cells were cultured as above for three days, harvested and stained for expression of B2M (ThermoFisher, MA5-18119) to verify presence of insertion and deletions (indels) and thus disruption of the open reading-frame of B2M.

### Results

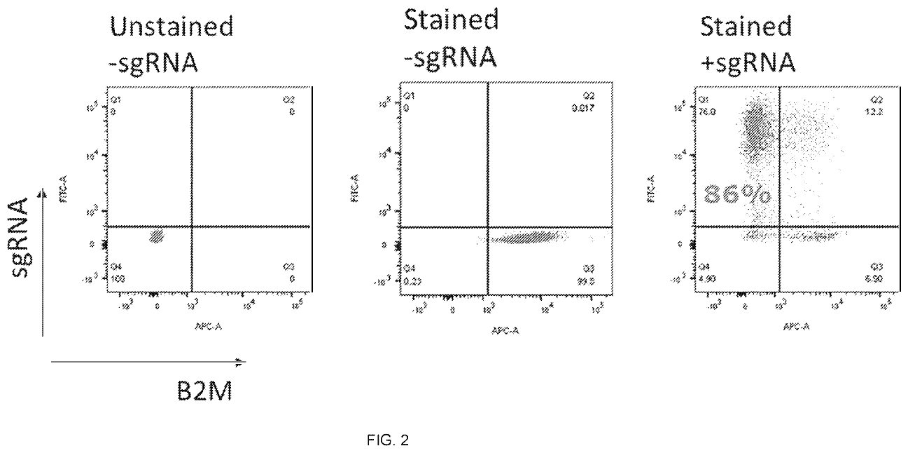

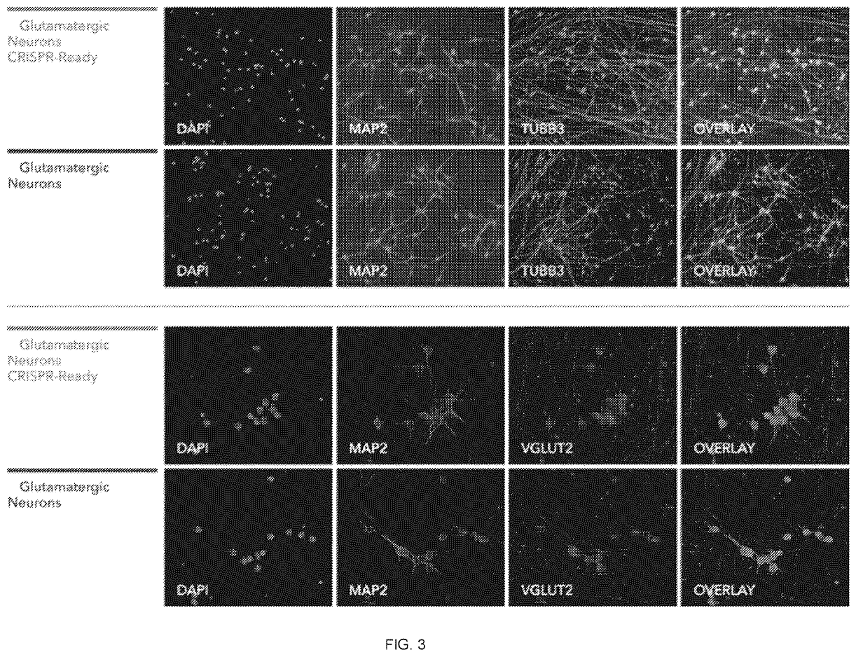

FIG. 2 shows the loss of B2M protein expression upon delivery of the specific sgRNA compared to an untransduced and unstained control. Loss was quite pronounced and observed in the majority of the cells that received a B2M-specific guide RNA (86% B2M-negative cells; gated on GFP positive cells). This confirms that Cas9 is active and activity is homogenous across the individual cells in the experiment.

### Example 3: Comparison of Glutamatergic Neurons and Glutamatergic Neurons CRISPR-Ready by Immunofluorescence Staining for Neuronal Markers

### Methods

To investigate potential variations in the protein expression profiles between “Glutamatergic Neurons” and “Glutamatergic Neurons CRISPR-Ready”, an immunofluorescence approach was utilized, focusing on three key neuronal markers: TUBB3, MAP2, and VGLUT2. The analysis was conducted on Day 11 after revival.

### Experimental Outline

Cells were seeded (Day −4) and cultured using media to support differentiation into Glutamatergic Neurons. During their reprogramming the cells underwent several media changes. At day 0 cells are cryopreserved. After revival, cells are cultured until day 11 for immunofluorescence staining.

Cells were fixed using 4% paraformaldehyde (ThermoFisher, 11586711) in DPBS (ThermoFisher, 14190144). Following fixation, a blocking solution containing either Donkey serum (Sigma-Aldrich, D9663) or Goat serum (Sigma-Aldrich, G9023) was applied. Cells were then labelled with primary antibodies: VGLUT2 (Millipore, MAB5504), MAP2 (Abcam, ab5392), or TUBB3 (Biolegend, 801202H). Post-primary labelling, secondary antibody labelling utilized DAPI (Biotechne, 5748/10) combined with either Donkey anti-mouse Alexa Fluor 488 (ThermoFisher, A-21202) or Goat anti-chicken Alexa Fluor 647 (ThermoFisher, A-21449). After labelling, cells were washed and imaged using a Leica DMi8 fluorescent microscope at 10× magnification.

### Results

Following a thorough analysis of the protein expression profiles of “Glutamatergic Neurons” and “Glutamatergic Neurons CRISPR-Ready” on Day 11 post-revival, the results revealed no significant differences in the expression of the neuronal markers TUBB3, MAP2, and VGLUT2. Both cell lines exhibit comparable staining intensities and patterns in the immunofluorescence assay, suggesting that stable Cas9 expression did not affect the cells' ability to differentiate into Glutamatergic Neurons (see FIG. 3).

### Example 4: Comparison of Glutamatergic Neurons and Glutamatergic Neurons CRISPR-Ready by Bulk RNA Sequencing to Assess Transcriptomic Differences

### Methods

To ensure that the expression of Cas9 doesn't induce disparities between “Glutamatergic Neurons” and “Glutamatergic Neurons CRISPR-Ready”, a comparative analysis was carried out. Leveraging Bulk RNA sequencing, the study aimed to discern any global transcriptomic level variations between the two cell lines. The assessment was executed at the iPSC stage, as well as on Day 4 and Day 18 post-revival.

### Experimental Outline

To obtain Glutamatergic neurons, cells were cultured as in Example 3, although this experiment is performed in continuous culture.

For each timepoint, experiments were conducted in triplicates to ensure reproducibility and accuracy. Total mRNA from these samples was isolated and libraries were prepared. The Bioinformatics analysis involved aligning the sequence reads to a reference genome, quantifying gene expression, and normalizing the data. Differentially expressed genes between the samples were then identified using established statistical models, offering insights into potential variations in the transcriptomic profiles.

### Results

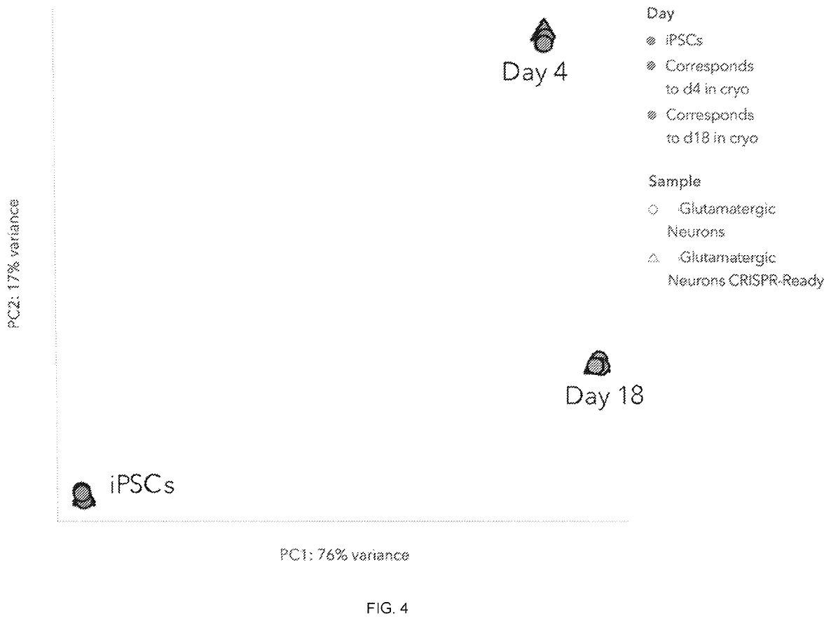

Utilizing bulk RNA sequencing data, a comprehensive transcriptomic analysis was performed to evaluate the potential transcriptomic differences between Cas9-expressing and non-expressing cells. Principal Component Analysis (PCA), a statistical method widely used to emphasize variation and bring out strong patterns in a dataset, was employed to elucidate the multidimensional transcriptomic differences. Results show that Glutamatergic Neurons CRISPR-Ready cluster closely to Glutamatergic Neurons at each time point analysed, indicating similar transcriptomic profiles between the two cell types (see FIG. 4). This consistency in global gene expression was evident across all three timepoints.

### Example 5: Western Blot Analysis of Cas9 Expression in Glutamatergic Neurons CRISPR-Ready Over Time

### Methods

Western blotting was employed to evaluate Cas9 expression in Glutamatergic Neurons throughout the process of forward programming and maturation.

### Experimental Outline

To obtain Glutamatergic neurons, cells were cultured as in Example 3, although this experiment is performed in continuous culture.

Western blot analysis was used to assess Cas9 expression over time. Cells are first harvested, washed in ice-cold PBS (Gibco, 20012027), and lysed using a RIPA buffer supplemented with protease inhibitors. After centrifugation to remove cell debris, the protein concentration in the supernatant is determined using the BCA assay. The proteins are then subjected to SDS-PAGE and transferred onto a methanol-activated PVDF membrane. The membrane is blocked and incubated with primary antibodies specifically targeting Cas9. β-actin is used as a loading control to ensure consistent sample application and validate the results. Following primary antibody incubation, the membrane is probed with secondary HRP-conjugated antibodies. Cas9 expression is visualized using the Amersham™ ECL™ Prime Western Blotting system.

### Results

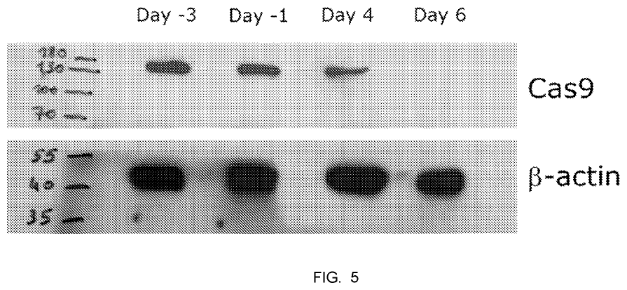

Analysis of the Western blot demonstrated a temporal decline in Cas9 expression across the examined time points (see FIG. 5). The differentiation process is structured in two phases: pre-differentiation and stabilisation/maturation. DO represents the day of cryopreservation. Days with a minus sign indicate the period of differentiation prior to preservation. Days without a minus sign represent the time after revival.

By Day 6, Cas9 expression was undetectable. This observed transient nature of Cas9 expression underscores the importance of timing in genome editing experiments. Taking these findings into account, the optimal temporal window for introducing guide RNA is between 24 to 72 hours after cell revival. Utilizing this window ensures the proficient formation of the Cas9-guide RNA complex, thereby facilitating the introduction of a precise double-strand break.

### Example 6: Amplicon Sequencing Analysis of Indel Formation in the SOX11 Gene

### Methods

To assess the functionality of Cas9 in forward-programmed cells, the knock-out efficiency of the SOX11 gene was examined at the DNA level using amplicon sequencing. The objective was to establish and optimize two distinct guide delivery methods: a lipid-based approach using RNAiMAX and a lentiviral delivery system.

### Experimental Outline

Guide RNAs were introduced into cells using both RNAiMAX and lentivirus on Day 1 and Day 3 post revival. Guide RNA sequence targeting SOX11: GAGAAGATCCCGTTCATCC (SEQ ID NO: 3). Guide RNA sequence non-targeting control: GCTACCCGCGCGAGAATTGC (SEQ ID NO: 4).

### Lipid-Based Delivery:

The synthetic guide RNA delivery protocol was adapted from the manufacturer's instructions provided with the Lipofectamine® RNAiMAX Reagent (ThermoFisher, 13778075). This protocol was designed with flexibility in mind to cater to varying applications. For the transfection, microcentrifuge tubes labelled A, B, and C were set up. Tube A combined DMEM/F-12 medium with sgRNA, while Tube B had a mix of DMEM/F-12 medium and the RNAiMAX reagent. The contents of Tubes A and B were combined into Tube C, mixed well by vortexing, and incubated at room temperature for 5 minutes. This resulting sgRNA-RNAIMAX mixture from Tube C was then evenly distributed in a drop-wise manner across the desired wells or dishes. Depending on the well size, the final concentrations of sgRNA when introduced to the cells were 2 μmol for 96-well plates, 10 μmol for 24-well plates, and 50 μmol for 6-well plates. Following this, plates were placed back in the incubator for 24 hours before the medium was refreshed based on the transfection timing.

### Lentiviral Delivery:

Lentivirus-containing supernatants were precipitated using the PEG-it™ Virus Precipitation Solution (System Biosciences, LV810A-1), strictly adhering to the manufacturer's directions. Transduction efficacy using lentivirus was assessed at 24 h and 72 h intervals post-cell revival. For the transduction process, the necessary virus volume per well or dish was calculated. This diluted lentivirus was then uniformly added drop-wise to the designated well or dish, ensuring even distribution by gentle agitation. Plates were then returned to the incubator for a 24-hour incubation. After this period, the medium was entirely refreshed based on the specific transduction timing. Regular cell culture maintenance was then continued, and the selected readout method was applied at the appropriate time.

Five days post guide delivery, genomic DNA was extracted from the treated cells. The targeted region within the SOX11 gene was amplified (primer 1: ACCCAGACTGGTGCAAGA (SEQ ID NO: 5); primer 2: GCTCTTCTCTGGGCTCTG (SEQ ID NO: 6)), indexed, and analysed using next-generation sequencing (NGS) to measure indel formation. A non-targeting guide served as a control for the experiment. For the bioinformatics analysis, raw sequencing reads were quality-checked and filtered. Using reference alignment, the reads were mapped to the SOX11 gene region. Indel events at the targeted sites were identified and quantified. The frequency of indels in samples treated with targeted guides was compared to the control to assess the specificity and efficiency of the edits introduced by the guide RNAs.

### Results

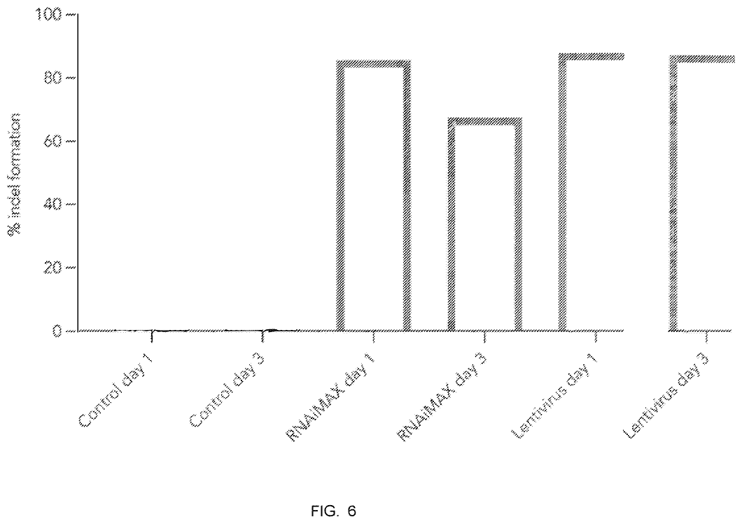

Analysis of the NGS data revealed a significant percentage of indel formation within the targeted region of the SOX11 gene (see FIG. 6). The efficiency of indel formation varied based on the guide delivery method and the timing of introduction. Specifically, indel formation ranged between 67% to 87%, depending on the condition. The non-targeting guide served as a control, confirming the specificity and efficiency of the edits introduced by the targeted guide RNAs.

### Example 7: Immunofluorescence Staining Analysis of SOX11 Expression Following Lipid-Based Guide RNA Delivery

### Methods

After confirming Cas9 functionality at the DNA level, the knock-out efficiency of SOX11 was further evaluated at the protein level using immunofluorescence staining. Image analysis was employed to directly assess the impact of genome editing on SOX11 protein expression post-guide RNA delivery using the lipid-based approach.

### Experimental Outline

gRNAs were delivered to revived cells as described in Example 6. By Day 11 post-revival, cells were used for immunofluorescence staining. To avoid cell detachment, they were fixed by exchanging half of the media volume with 8% Paraformaldehyde (PFA) from ThermoFisher (Cat. No. 11586711), followed by a second fixation step with 4% PFA only. After multiple PBS (Gibco, 20012027) washes, cells were incubated with primary antibody (SOX11 Monoclonal Antibody from ThermoFisher, 14-9773-82) overnight at 4° C. Post-wash, secondary antibodies and DAPI (Biotechne, 5748/10) were incubated for an hour. Following a series of washes with 0.1% Triton-X (Sigma, T8787), cells were stored in PBS until their subsequent analysis. To assess the knock-out efficiency, an advanced image analysis pipeline was employed. This pipeline incorporated Deep Learning methodologies, specifically tailored for precise recognition of cell nuclei. Utilizing this approach, the intensity and distribution of the DAPI signal, indicative of cell nuclei, as well as the SOX11 signal, were meticulously quantified. The derived data, based on the correlation between DAPI and SOX11 signals, provided an accurate measure of the knock-out efficiency of SOX11 at the protein level.

### Results

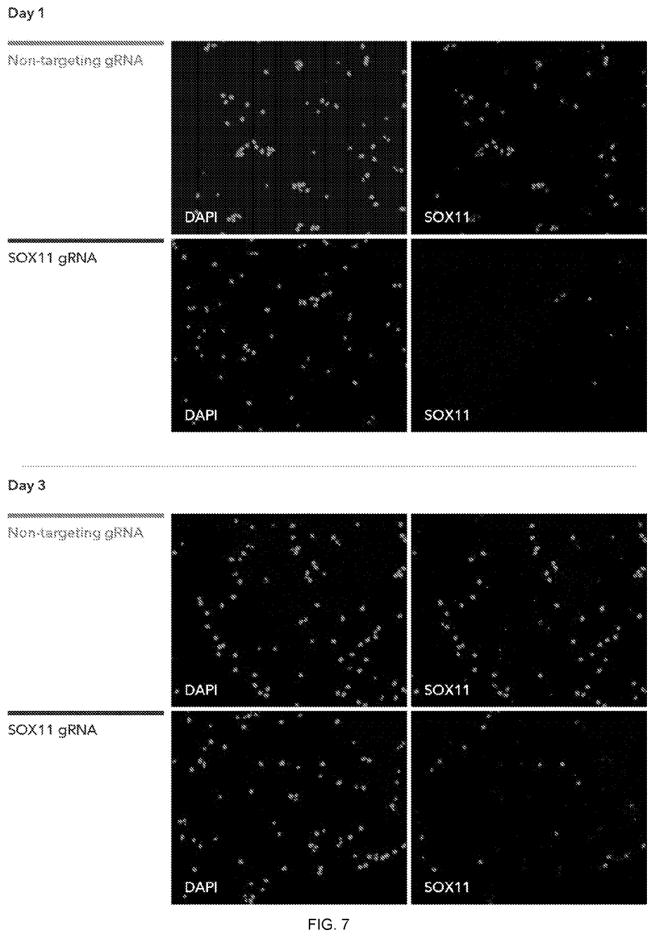

Guide RNA was delivered to cells using RNAiMAX on Day 1 and Day 3. By Day 11, SOX11 expression was examined with immunofluorescence staining. DAPI staining marked the cell nuclei, and a non-targeting guide was used as a control. Image analysis indicated a significant knock-out of SOX11 protein following introduction of the SOX11-specific gRNA (see FIG. 7).

### Example 8: Immunofluorescence Staining Analysis of SOX11 Expression Following Lentiviral Guide RNA Delivery

### Methods

After confirming Cas9 functionality at the DNA level, the knock-out efficiency of SOX11 was further evaluated at the protein level using immunofluorescence staining. Image analysis was employed to directly assess the impact of genome editing on SOX11 protein expression post lentiviral guide RNA delivery.

### Experimental Outline

Experimental outline is described in Example 7. Guide RNAs were delivered by lentiviral transduction as explained in Example 6.

### Results

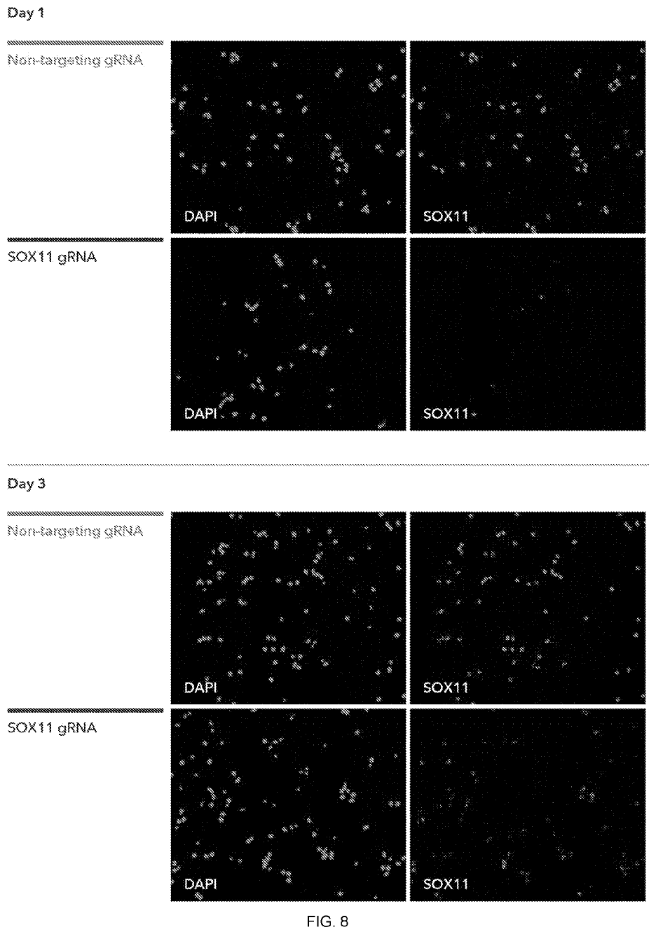

Guide RNA was delivered to cells using lentiviral transduction on Day 1 and Day 3. By Day 11, SOX11 expression was examined with immunofluorescence staining. DAPI staining marked the cell nuclei, and a non-targeting guide was used as a control. Image analysis indicated a significant knock-out of SOX11 protein following introduction of the SOX11-specific gRNA (see FIG. 8).

### Example 9: UMAP Visualization from a Pooled scCRISPR Screen Demonstrating Transcriptomic Similarity Among Specific Gene Knock-Outs

### Methods

The primary objective of this experiment was to illustrate the use of “Glutamatergic Neurons CRISPR-Ready” technology for pooled single-cell CRISPR (scCRISPR) screens on a panel of 100 genes associated with neurodegenerative diseases.

### Experimental Outline

Plasmid library encoding the CRISPR Guide RNAs for 100 genes involved in neurodegenerative diseases:

For each gene, four guide RNAs were designed and synthesized including non-targeting and gene desert controls. The guide RNAs were cloned in a pooled fashion into a lentiviral vector. The used vector encodes a puromycin-resistance cassette for the selection of cells with successful genomic integration. In addition, the vector contains GFP which is used to estimate multiplicity of infection. The quality of the library was assessed using next generation sequencing. Cell culture scale and transduction parameters were optimized to ensure an adequate coverage.

### Screening Outline:

Prior to transduction, Glutamatergic neurons were revived as described in Example 6. Following transduction on day 3, cells were cultured and selected for successful genomic integration by adding puromycin to the culture media. Following selection, cells were harvested, and single cell dissociated. Dissociated single cells were processed on a 10× Genomics Chromium machine.

Single-Cell Transcriptome Analysis and sgRNA Capture:

Glutamatergic neurons were analysed by scRNA-seq using 10× Genomics Chromium Single Cell 5′ Reagent Kits v2 following manufacturer's instructions. Guide RNA was captured by spiking in a guide capture oligo as described in literature (Replogle et al., Nature Biotechnology 2020). 10233 cells were retrieved from the 10× Genomics Chromium X machine. After the cDNA amplification step, gene expression and guide RNA libraries were created and sequenced on a NovaSeq sequencer.

### Data Analysis:

Single-cell gene expression data processed by the STARsolo (Kaminow et al., bioRxiv, 2021) tool were further analysed in Seurat v4 (Hao et al., Cell, 2021) and visualized on uniform manifold approximation and projection (UMAP) plots (Becht, Nature Biotech, 2019).

### Results

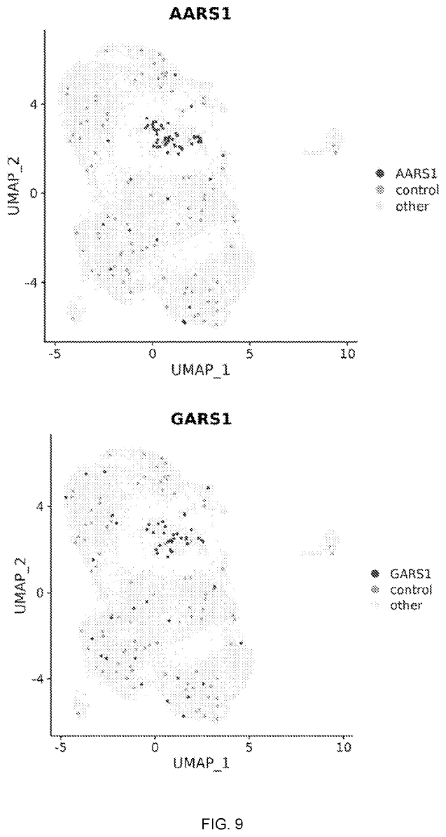

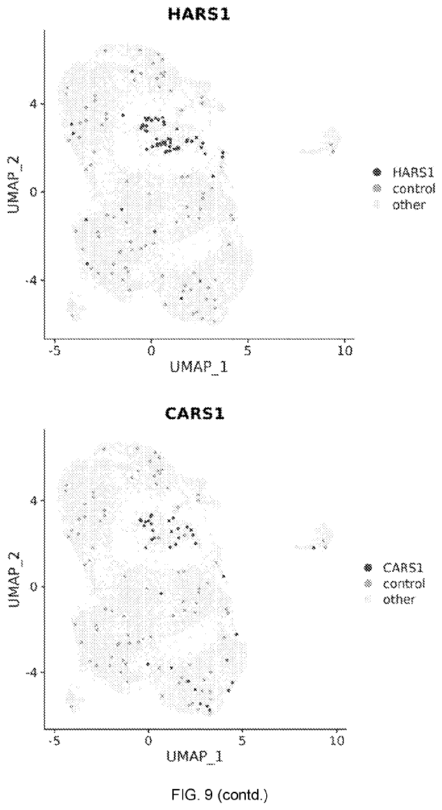

The single-cell transcriptomic analysis of gene knockouts in forward programmed Glutamatergic neurons unveiled distinct transcriptomic phenotypes resulting from the disruption of disease-linked genes. Notably, cells with AARS1, CARS1, HARS1, and GARS1 knock-outs clustered together, confirming the effective perturbation of these genes (see FIG. 9). These aminoacyl-tRNA synthetases (aaRSs) have pivotal roles in the initial stages of protein biosynthesis, facilitating the precise charging of transfer RNAs (tRNAs) with their corresponding amino acids. Beyond their critical role in protein synthesis, these enzymes also contribute to protein quality control and proper protein folding within the endoplasmic reticulum (ER). It is worth noting that loss-of-function mutations in these genes have been reported in Charcot-Marie-Tooth disease patients, underscoring their clinical relevance and implicating them in the pathogenesis of this neurodegenerative disorder. These findings not only shed light on cellular processes and their relevance to Charcot Marie Tooth disease but also demonstrate the potential of using these cells to discover meaningful biology.

### Example 10: Gene Engineering Strategy for Cas9 Expressing Microglia

### Methods

The aim of this experiment was to create an iPSC line that serves two purposes: (i) it can be forward programmed by the overexpression of transcription factors (e.g. SPI1-CEBPB for Microglia) and (ii) it expresses the Cas9 protein in a constitutive manner to enable gene editing in forward programmed/differentiated cells. Thereto, three transgenes were integrated into genome safe-harbour sites (GSH) by genome engineering. Firstly, a CAG promoter-driven reverse Tet transactivator (rtTA) cassette for doxycycline-controlled transactivation of a second recombinant transgene was integrated into the ROSA26 locus. Secondly, transcription factor(s) under the control of doxycycline-responsive promoter was integrated into the AAVS1 locus. Lastly, the CAG promoter-driven full-length Cas9 protein was integrated into the CLYBL locus, upstream of a P2A ribosomal-skipping sequence and a fluorescent protein for verification of integration.

### Experimental Outline

An iPSC line was cultured at all times. Editing of the safe harbour loci was performed subsequentially. All three loci were edited using CRISPR/Cas9 technology. A sgRNA which introduces a DNA double-strand break in close proximity to the chosen insertion site was selected. Cells were nucleofected with two plasmids—a plasmid expressing Cas9 and the corresponding sgRNA and a plasmid containing the insert sequence flanked by homology arms. The PAM sequence was mutated to prevent the re-cutting of Cas9.

Cells harbouring the correct integration were selected (e.g. using antibiotic resistance markers with the respective antibiotic). Once selection has reached a sufficient efficiency, cells were dissociated as single cells. To obtain monoclonal lines, cells were seeded at a low density to allow outgrowth of colonies originating from a single cell.

Engineered lines were submitted to two rounds of genotyping by PCR. The first round of genotyping was performed on crude lysate. Once clones with the correct integration were identified, cells were expanded, and genomic DNA was isolated for a second round of genotyping. PCRs for correct integration of 5′ and 3′ homology arms as well as the insert sequence. In addition, we performed PCRs to confirm if clones are heterozygous or homozygous for the intended integration.

### Results

The final iPSC line has been engineered to incorporate the dual safe harbour system, allowing transcription factor overexpression. It contains transcription factor(s) essential for forward programming into Microglia. Additionally, this line constitutively expresses the Cas9 protein, facilitating further genomic manipulations.

### Example 11: Comparison of Transcriptomic Profiles Between Microglia and Microglia CRISPR-Ready

### Methods

To ensure that the expression of Cas9 does not induce differences between Microglia and Microglia CRISPR-Ready, a comparative analysis was carried out. Leveraging Bulk RNA sequencing, the study aimed to discern any global transcriptomic level variations between the two cell lines. The assessment was executed at the iPSC stage, as well as on Day 10 (before cryopreservation) and Day 20 (Day 10 post cryopreservation).

### Experimental Outline

After thawing, cells were cultured using media to support differentiation into Microglia. During their reprogramming the cells underwent several media changes.

For each timepoint, experiments were conducted in triplicates to ensure reproducibility and accuracy. Total mRNA from these samples was isolated and libraries were prepared. The Bioinformatics analysis involved aligning the sequence reads to a reference genome, quantifying gene expression, and normalizing the data. Differentially expressed genes between the samples were then identified using established statistical models, offering insights into potential variations in the transcriptomic profiles.

### Results

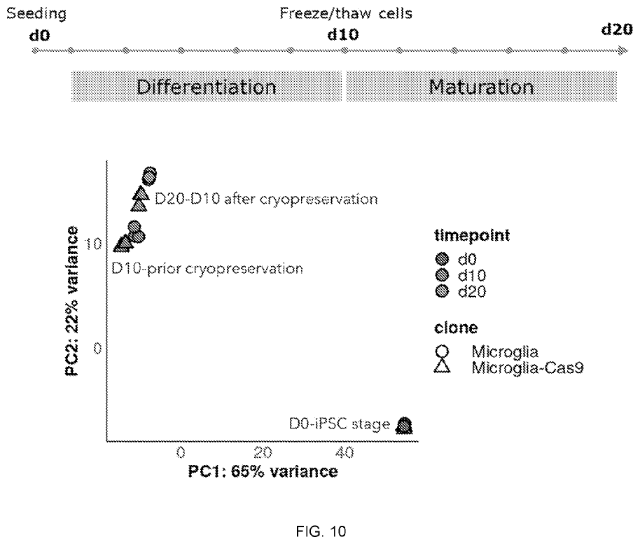

Utilizing bulk RNA sequencing data, a comprehensive transcriptomic analysis was performed to evaluate the potential transcriptomic differences between Cas9-expressing and non-expressing cells. Principal Component Analysis (PCA), a statistical method widely used to emphasize variation and bring out strong patterns in a dataset, was employed to elucidate the multidimensional transcriptomic differences. Results show that Microglia CRISPR-Ready cluster closely to Microglia at each time point analysed, indicating similar transcriptomic profiles between the two cell types (see FIG. 10). This consistency in global gene expression was evident across all three timepoints.

### Example 12: Comparison of Secreted Cytokine Profiles Between Microglia and Microglia CRISPR-Ready Upon LPS Stimulation

### Methods

To ensure that the expression of Cas9 does not induce differences between Microglia and Microglia CRISPR-Ready activation states, a comparative analysis was carried out using the Proteome Profiler Human Cytokine Array kit (R&D Systems, ARY005B)).

### Experimental Outline

Cells were cultured following the procedure outlined in Example 11. On the ninth day after revival, they were subjected to a treatment with 100 ng/ml of Lipopolysaccharide (LPS) (R&D Systems, LPS25). After a 24-hour incubation period, the supernatants were collected and analysed for cytokine release using the Proteome Profiler Human Cytokine Array kit (ARY005B, R&D Systems).

### Results

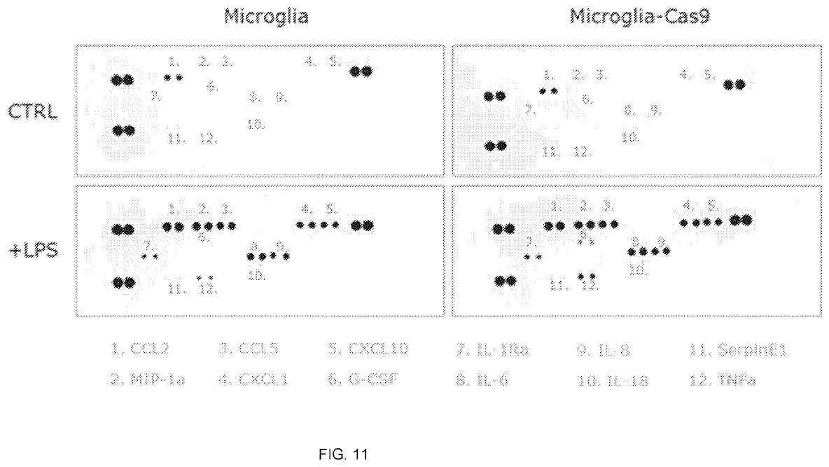

The results indicate that there were no discernible differences in cytokine secretion between Microglia and Microglia CRISPR-ready cells (see FIG. 11). Both cell types exhibited clear activation in response to LPS stimulation, suggesting similar cytokine secretion profiles in both cases.

### Example 13: Benchmarking of Cas9 Expression and Activity in Forward Programmed Microglia

### Methods

To measure the activity of Cas9 in Microglia at D9 (after cryopreservation), a guide RNA targeting the B2 microglobulin (B2M) gene (GGCCGAGATGTCTCGCTCCG, SEQ ID NO: 2), which is expressed on all nucleated cells, was cloned into a lentiviral vector. The used vector encodes a puromycin-resistance cassette for the selection of cells with successful genomic integration. In addition, the vector contains GFP in order to estimate the multiplicity of infection. The lentiviral vector was used to transduce cells harbouring an rtTA transactivator, constitutive full-length Cas9 and the transcription factor combination necessary for forward programming under the control of a Tet-responsive promoter.

### Experimental Outline

Cells were cultured following the procedure outlined in Example 11. On day 9 after revival, cells were transduced with the lentiviral vector targeting B2M. Following transduction, cells were cultured as above for 5 days, harvested and stained for expression of B2M to verify presence of insertion and deletions (indels) and thus disruption of the open reading-frame of B2M. In addition, cells were stained for CD11b which is a marker of microglial cells.

### Results

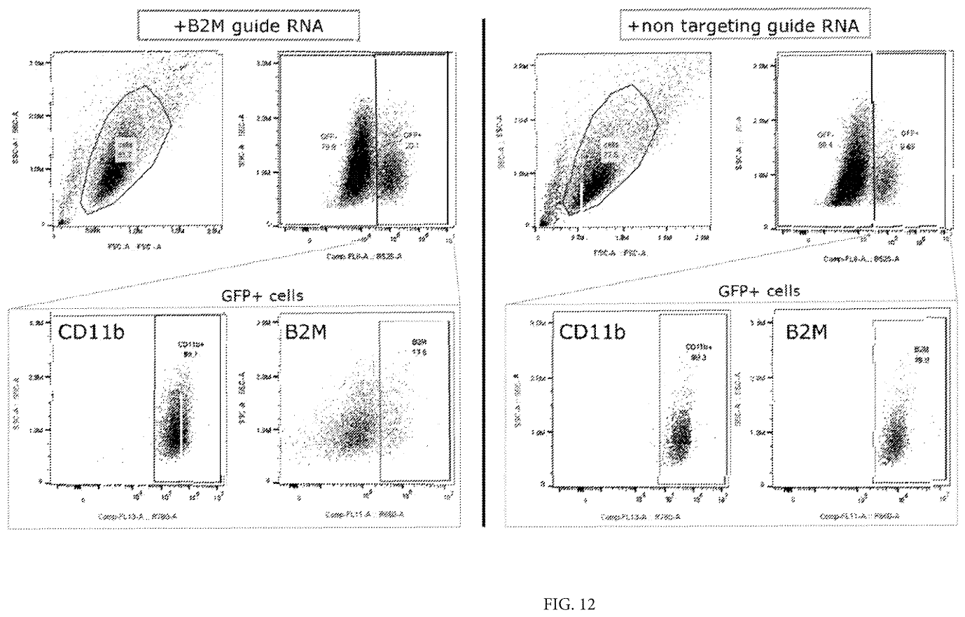

FIG. 12 shows the loss of B2M protein expression upon delivery of the specific sgRNA compared to a non-targeting control. Loss was quite pronounced and observed in the majority of the cells that received a B2M-specific guide RNA (86.4% B2M-negative cells; gated on GFP positive cells). This confirms that Cas9 is active and activity is homogenous across the individual cells in the experiment. Cells remain positive for CD11b upon lentiviral transduction.

### Example 14: Benchmarking of Cas9 Expression and Activity in Forward Programmed Microglia Over Time

### Methods

To measure the activity of Cas9 in Microglia at D1, D6, D9 and D18 (after cryopreservation), a guide RNA targeting the 32 microglobulin (B2M) gene (GGCCGAGATGTCTCGCTCCG, SEQ ID NO: 2), which is expressed on all nucleated cells, was cloned into a lentiviral vector. The used vector encodes a puromycin-resistance cassette for the selection of cells with successful genomic integration. In addition, the vector contains GFP in order to estimate the multiplicity of infection. The lentiviral vector was used to transduce cells harbouring an rtTA transactivator, constitutive full-length Cas9 and the transcription factor combination necessary for forward programming under the control of a Tet-responsive promoter.

### Experimental Outline

Cells were cultured following the procedure outlined in Example 11. On day 1, day 6, day 9 and day 18 after revival, cells were transduced with the lentiviral vector targeting B2M. Following transduction, cells were cultured as above for 5 days, harvested and stained for expression of B2M (ThermoFisher, MA5-18119) to verify presence of insertion and deletions (indels) and thus disruption of the open reading-frame of B2M. In addition, cells are stained for CD11b which is a marker of microglial cells.

### Results

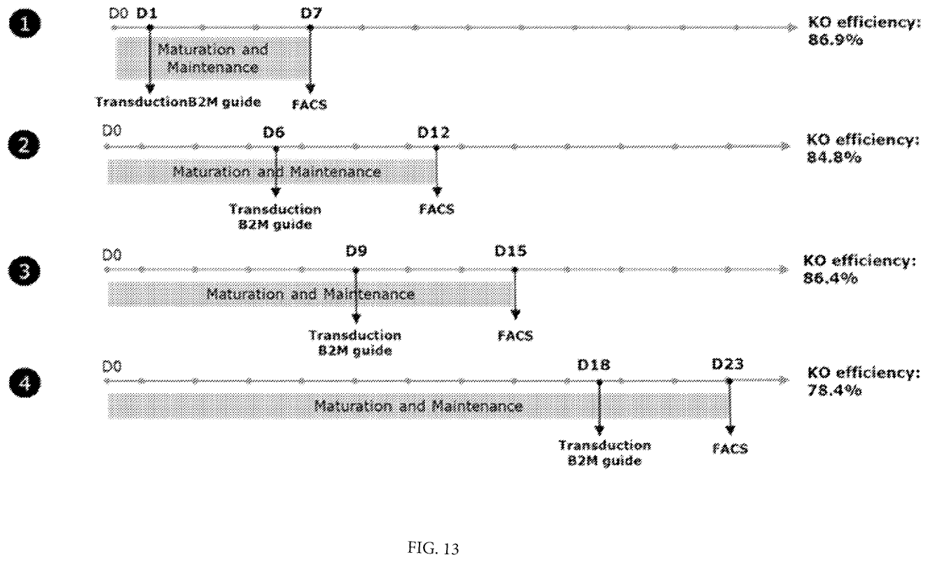

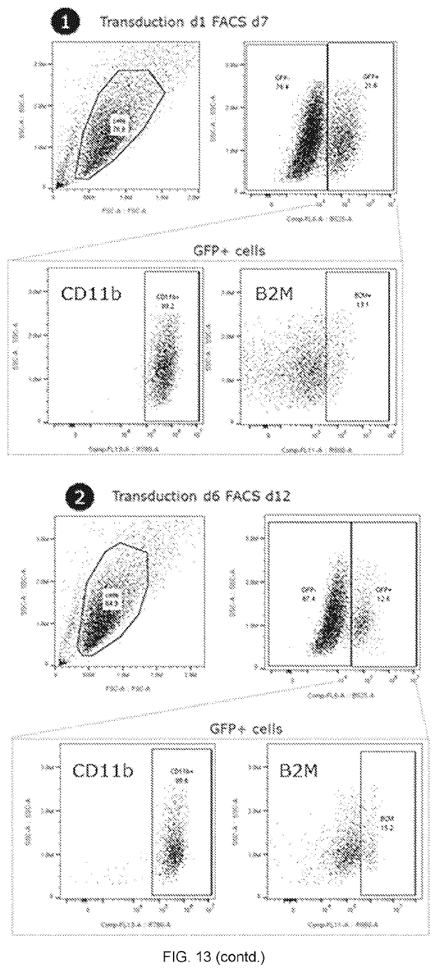

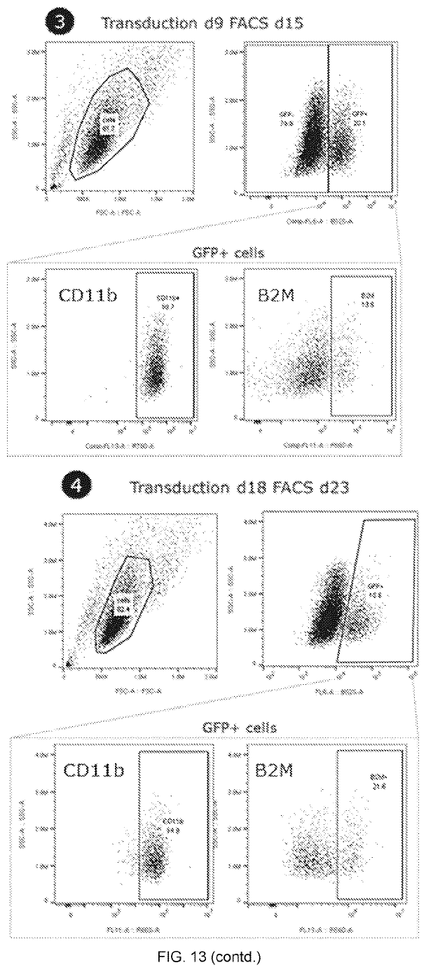

Flow cytometry analysis demonstrates a clear decrease in B2M protein levels after delivering the B2M sgRNA (see FIG. 13). This reduction is consistent across most cells that received the B2M-specific guide RNA (gated on GFP positive cells; Day 1:86.9%, Day 6:84.8%, Day 9: 86.4%, Day 18:78.4%), indicating that Cas9 is active and it remains expressed over time. Importantly, this example highlights that Cas9 can withstand silencing which is a common problem. Cells transduced with a gRNA remain CD11b positive.

### Example 15: ScCRISPR Screen for Modulators of Microglial Activation Upon LPS Stimulation

### Methods

The primary objective of this experiment was to illustrate the use of “Microglia CRISPR-Ready” cells for pooled single-cell CRISPR (scCRISPR) screens on a panel of 110 genes associated with neurodegenerative diseases and microglial activation states.

### Experimental Outline

Plasmid library encoding the CRISPR Guide RNAs for 110 genes involved in neurodegenerative diseases and microglial activation states:

For each gene, four guide RNAs were designed and synthesized including non-targeting and gene desert controls. The guide RNAs were cloned in a pooled fashion into a lentiviral vector. The used vector encodes a puromycin-resistance cassette for the selection of cells with successful genomic integration. In addition, the vector contains GFP which is used to estimate multiplicity of infection. The quality of the library was assessed using next generation sequencing. Cell culture scale and transduction parameters were optimized to ensure an adequate coverage.

### Screening Outline:

Prior to transduction, Microglia were revived as described in Example 11. Following transduction on day 9, half the plates of cells were stimulated with LPS (100 ng/ml) and the other half was left untreated. After 24 h cells were dissociated and GFP cells were sorted using a cell sorter machine. Sorted single cells were processed on a 10× Genomics Chromium machine.

Single-Cell Transcriptome Analysis and sgRNA Capture:

Microglia were analysed using a targeted sequencing readout. scRNA-seq was performed using 10× Genomics Chromium Single Cell 3′ Reagent Kits v2 following manufacturer's instructions. Approx. 30.000 cells were retrieved per condition (+/−LPS from the 10× Genomics Chromium X machine. Primer panels designed to capture the activation signature determined using bulk RNA-seq were pooled and used for library preparation (Schraivogel et al. Nature methods 2020). After the cDNA amplification step, gene expression and guide RNA libraries were created and sequenced on a NovaSeq sequencer.

### Data Analysis:

Single-cell gene expression data processed by the STARsolo (Kaminow et al., bioRxiv, 2021) tool were further analysed in Seurat v4 (Hao et al., Cell, 2021) and visualized on cosine similarity plots.

### Results

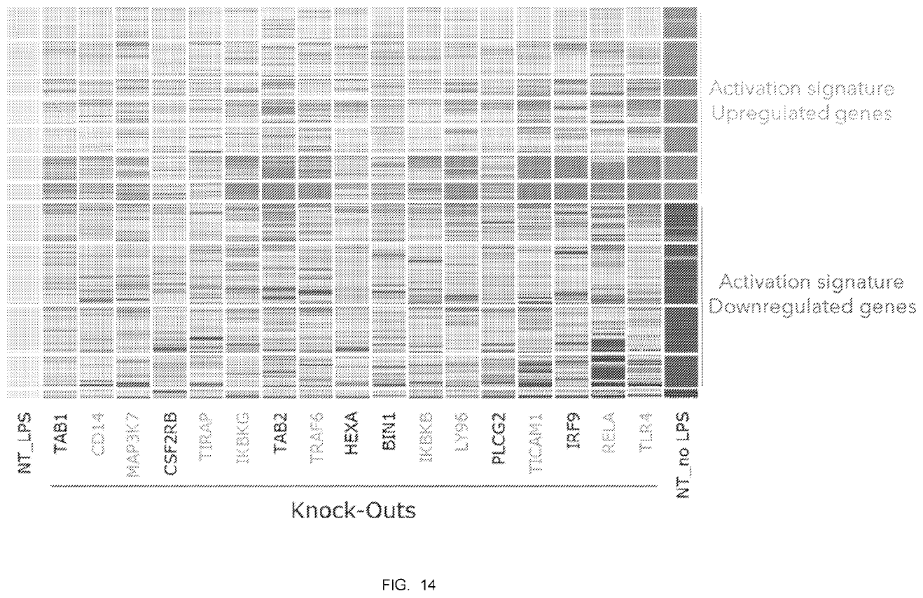

FIG. 14 illustrates a heatmap highlighting genes identified as an activation signature in this study. These genes were discerned through the analysis of bulk RNA-seq data derived from cells subjected to both LPS treatment (+LPS) and those left untreated (−LPS), with the genes arranged along the y-axis. To assess the similarity between different conditions, Cosine Similarity Analysis was employed, utilizing non-targeting guides from both the LPS-treated (+LPS) and untreated (−LPS) conditions. On the x-axis, knockout mutations in the LPS-treated condition are plotted, indicating cells that exhibited a higher resemblance to the untreated condition without LPS, specifically those with non-targeting guides.

Notably, certain genes within the heatmap are shaded in grey, indicating their association with the Toll-like Receptor 4 (TLR4) pathway. This pathway is known for its pivotal role in initiating microglial activation in response to LPS treatment. In summary, FIG. 14 provides a visual representation of genes comprising an activation signature and their response to LPS treatment, elucidated through cosine similarity analysis. Furthermore, it underscores the significance of genes linked to the TLR4 pathway in the context of microglial activation triggered by LPS treatment, affirming the utility of CRISPR-ready cells employed in a scCRISPR screen for generating biologically meaningful data.

### Example 16: Gene Engineering Strategy for dCas9-VPR Expressing Glutamatergic Neurons

### Methods

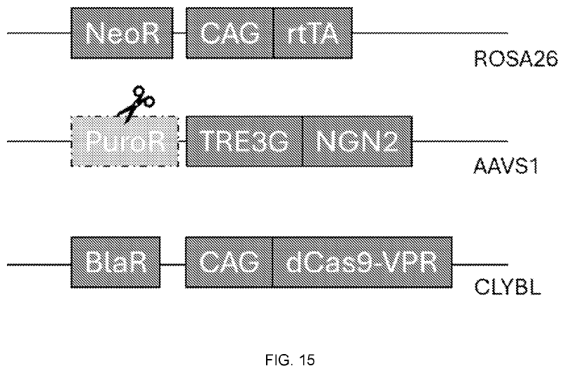

The aim of this experiment was to create an iPSC line that serves two purposes: (i) it can be forward programmed by the overexpression of transcription factors (e.g. NEUROG2 (NGN2) for Glutamatergic Neurons) and (ii) it expresses the dCas9-VPR protein in a constitutive manner to enable gene activation (CRISPRa) in forward programmed/differentiated cells. Thereto, three transgenes were integrated into genome safe-harbour sites (GSH) by genome engineering (see FIG. 15). Firstly, a CAG promoter-driven reverse Tet transactivator (rtTA) cassette for doxycycline-controlled transactivation of a second recombinant transgene was integrated into the ROSA26 locus. Secondly, a transcription factor (e.g. NGN2) under the control of doxycycline-responsive promoter was integrated into the AAVS1 locus. Lastly, the CAG promoter-driven dCas9-VPR protein was integrated into the CLYBL locus.

### Experimental Outline

An iPSC line was cultured at all times. Editing of the safe harbour loci was performed subsequentially. All three loci were edited using CRISPR/Cas9 technology. A sgRNA which introduces a DNA double-strand break in close proximity to the chosen insertion site was selected. Cells were nucleofected with two plasmids—a plasmid expressing Cas9 and the corresponding sgRNA and a plasmid containing the insert sequence flanked by homology arms. The PAM sequence was mutated to prevent the re-cutting of Cas9. Cells harbouring the correct integration were selected (e.g. using antibiotic resistance markers with the respective antibiotic). Once selection has reached a sufficient efficiency, cells were dissociated as single cells. To obtain monoclonal lines, cells were seeded at a low density to allow outgrowth of colonies originating from a single cell. Engineered lines were submitted to two rounds of genotyping by PCR. The first round of genotyping was performed on crude lysate. Once clones with the correct integration were identified, cells were expanded, and genomic DNA was isolated for a second round of genotyping. PCRs for correct integration of 5′ and 3′ homology arms as well as the insert sequence were conducted. In addition, PCRs were performed to confirm if clones are heterozygous or homozygous for the intended integration.

### Results

The final iPSC line has been engineered to incorporate the dual safe harbour system, allowing transcription factor overexpression. It contains transcription factor(s) essential for forward programming into Glutamatergic neurons. Additionally, this line constitutively expresses the dCas9-VPR protein, facilitating CRISPR activation experiments.

### Example 17: Benchmarking of dCas9-VPR Expression and Activity at iPSC Stage

To measure the activity of Cas9 at iPSC stage, a guide RNA targeting the CD274 gene (GTCAGGAAAGTCCAACGCC, SEQ ID NO: 7), which is lowly expressed in iPSCs and ioGlutamatergic neurons, was cloned into a lentiviral vector. The used vector encodes a puromycin resistance cassette for the selection of cells with successful genomic integration. In addition, the vector contains GFP in order to estimate the multiplicity of infection. The lentiviral vector was used to transduce iPSCs harbouring an rtTA transactivator, constitutive dCas9-VPR and the transcription factor NGN2 necessary for forward programming under the control of a Tet-responsive promoter.

### Experimental Outline

Prior to lentiviral transduction, iPSCs were expanded on standard tissue culture plates. Cells were transduced with the lentiviral vector targeting CD274. Following transduction, cells were cultured as above for three days, harvested and stained for expression of CD274 (BD Bioscience, 563741) to verify the activation of CD274.

### Results

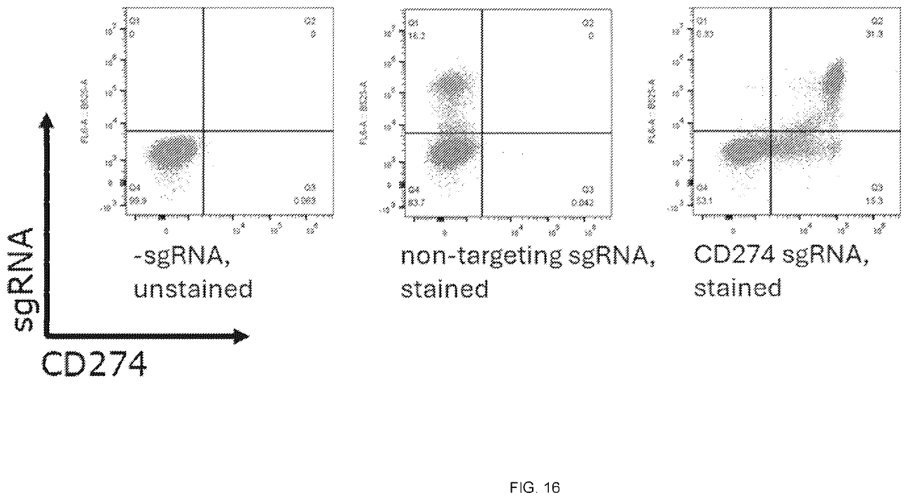

FIG. 16 shows the activation of CD274 protein expression upon delivery of the specific sgRNA compared to a non-transduced, unstained and non-targeting control. Activation was observed in the majority of the cells that received a CD274-specific guide RNA (98.96% of cells showing activation; gated on GFP positive cells). This confirms that dCas9-VPR is functional.

### Example 18: Benchmarking of dCas9-VPR Functionality in ioGlutamatergic Neurons

### Methods

After confirming dCas9-VPR functionality at the iPSC stage, the gene activation efficiency of CD274 was further evaluated in differentiated cells. Flow-based analysis was employed to directly assess the impact of gene activation of CD274 protein expression post lentiviral guide RNA delivery.

### Experimental Outline

Lentiviral guide RNA delivery: Lentivirus-containing supernatants were precipitated using the PEG-it™ Virus Precipitation Solution (System Biosciences, LV810A-1), strictly adhering to the manufacturer's directions. Transduction efficacy using lentivirus was assessed at day 1, day 3 and day 11 post-cell revival. The diluted lentivirus was added dropwise to the designated well or dish, ensuring even distribution by gentle agitation. Plates were then returned to the incubator for a 24-hour incubation. After this period, the medium was entirely refreshed based on the specific transduction timing. Regular cell culture maintenance was continued, and the selected readout method was applied at the respective timepoints. Three days post guide delivery, a flow-based readout was used to assess CD274 activation. For this purpose, cells were single cell dissociated and stained with a CD274 antibody. After incubation and washing steps, the signal of CD274 was measured using a FACS machine. Non-transduced and unstained cells as well as cells with a non-targeting guide RNA were used as controls.

### Results

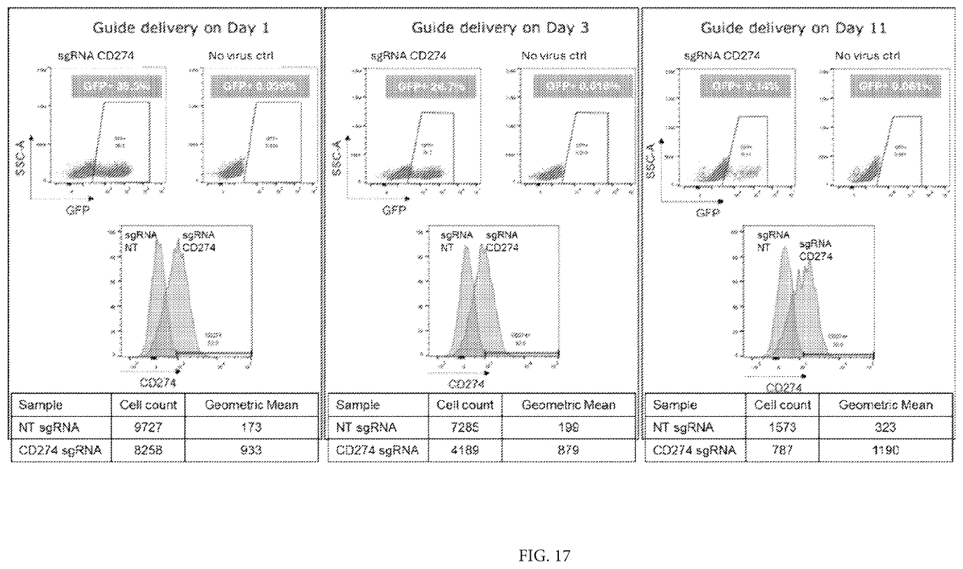

Guide RNA was delivered to cells using lentiviral transduction on day 1, day 3 and day 11. Three days post guide RNA delivery, CD274 expression was examined with a flow-based readout. FIG. 17 shows the activation of CD274 protein expression upon delivery of the specific guide RNA compared to a non-transduced, unstained and non-targeting control. Activation was observed in the majority of the cells that received a CD274-specific guide RNA (Mean Fluorescence Intensity (MFI) Day 1:5.4× increase; MFI Day 3:4.4× increase; MFI Day 11:3.7× increase; gated on GFP positive cells). This confirms that dCas9-VPR is functional in forward programmed cells.

### Example 19: Gene Engineering Strategy for dCas9-ZIM3 Expressing Glutamatergic Neurons

### Methods

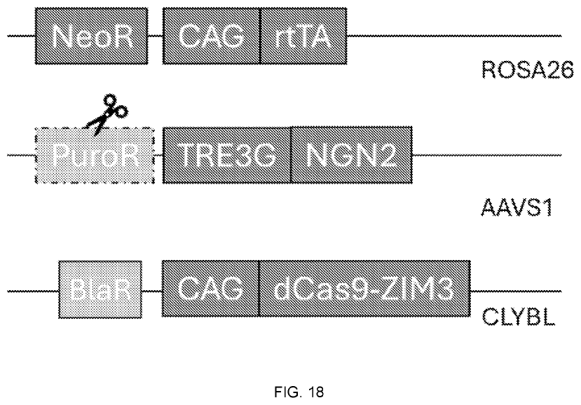

The aim of this experiment was to create an iPSC line that serves two purposes: (i) it can be forward programmed by the overexpression of transcription factors (e.g. NEUROG2 (NGN2) for Glutamatergic Neurons) and (ii) it expresses the dCas9-ZIM3 protein in a constitutive manner to enable gene inhibition (CRISPRi) in forward programmed/differentiated cells. Thereto, three transgenes were integrated into genome safe-harbour sites (GSH) by genome engineering (see FIG. 18). Firstly, a CAG promoter-driven reverse Tet transactivator (rtTA) cassette for doxycycline-controlled transactivation of a second recombinant transgene was integrated into the ROSA26 locus. Secondly, a transcription factor (e.g. NGN2) under the control of doxycycline-responsive promoter was integrated into the AAVS1 locus. Lastly, the CAG promoter-driven dCas9-ZIM3 protein was integrated into the CLYBL locus.

### Experimental Outline

An iPSC line was cultured at all times. Editing of the safe harbour loci was performed subsequentially. All three loci were edited using CRISPR/Cas9 technology. A sgRNA which introduces a DNA double-strand break in close proximity to the chosen insertion site was selected. Cells were nucleofected with two plasmids—a plasmid expressing Cas9 and the corresponding sgRNA and a plasmid containing the insert sequence flanked by homology arms. The PAM sequence was mutated to prevent the re-cutting of Cas9. Cells harbouring the correct integration were selected (e.g. using antibiotic resistance markers with the respective antibiotic). Once selection has reached a sufficient efficiency, cells were dissociated as single cells. To obtain monoclonal lines, cells were seeded at a low density to allow outgrowth of colonies originating from a single cell. Engineered lines were submitted to two rounds of genotyping by PCR. The first round of genotyping was performed on crude lysate. Once clones with the correct integration were identified, cells were expanded, and genomic DNA was isolated for a second round of genotyping. PCRs for correct integration of 5′ and 3′ homology arms as well as the insert sequence were conducted. In addition, PCRs were performed to confirm if clones are heterozygous or homozygous for the intended integration.

### Results

The final iPSC line has been engineered to incorporate the dual safe harbour system, allowing transcription factor overexpression. It contains transcription factor(s) essential for forward programming into Glutamatergic neurons. Additionally, this line constitutively expresses the dCas9-ZIM3 protein, facilitating CRISPRi experiments.

### Example 20: Benchmarking of dCas9-ZIM3 Functionality in ioGlutamatergic Neurons

### Methods

The efficiency of SOX11 gene inhibition was assessed in ioGlutamatergic neurons expressing dCas9-ZIM3 by Immunofluorescence staining for SOX11. Image analysis was employed to directly assess the impact of CRISPRi on SOX11 protein expression post lentiviral guide RNA delivery.

### Experimental Outline

The design criteria for CRISPRi guide RNAs are less well understood compared to those for CRISPR knockout experiments. To address this, three distinct CRISPRi guide RNAs were designed to target the SOX11 gene. After subcloning the single guide RNAs into a lentiviral vector, lentiviral particles are generated. Lentivirus-containing supernatants were precipitated using the PEG-it™ Virus Precipitation Solution (System Biosciences, LV810A-1), strictly adhering to the manufacturer's instructions. Guides were delivered in two different dilutions (1:500 and 1:1000) to revived cells on day 3. By Day 8 post-revival, cells were used for immunofluorescence staining. To avoid cell detachment, they were fixed by exchanging half of the media volume with 8% Paraformaldehyde (PFA; ThermoFisher, 11586711), followed by a second fixation step with 4% PFA only. After multiple PBS (Gibco, 20012027) washes, cells were incubated with primary antibody (SOX11 Monoclonal Antibody from ThermoFisher, 14-9773-82) overnight at 4° C. Post-wash, secondary antibodies and DAPI (Biotechne, 5748/10) were incubated for an hour. Following a series of washes with 0.1% Triton-X (Sigma, T8787), cells were stored in PBS until their subsequent analysis. To assess the knock down efficiency, an advanced image analysis pipeline was employed. This pipeline incorporated Deep Learning methodologies, specifically tailored for precise recognition of cell nuclei. Utilizing this approach, the intensity and distribution of the DAPI signal, indicative of cell nuclei, as well as the SOX11 signal, were meticulously quantified. The derived data, based on the correlation between DAPI and SOX11 signals, provided an accurate measure of the knock down efficiency of SOX11 at the protein level.

### Results

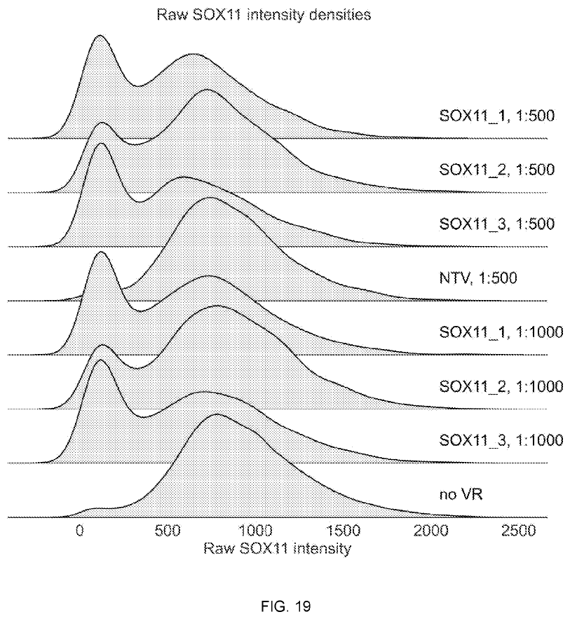

Three different guide RNA targeting SOX11 were delivered to cells using lentiviral transduction on day 3. Five days post guide RNA delivery, SOX11 expression was examined by immunofluorescence staining. FIG. 19 shows decrease of SOX11 protein expression upon delivery of the specific guide RNAs compared to a non-transduced (no VR), and non-targeting control (NTV). Inhibition was observed in the majority of the cells that received a SOX11-specific guide RNA. This confirms that dCas9-ZIM3 is functional in differentiated cells.

### Example 21: Gene Engineering Strategy for Cas9 Expressing ioOligodendrocyte-Like Cells

### Methods

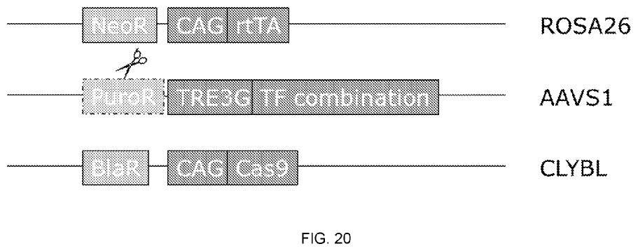

The aim of this experiment was to create an iPSC line that serves two purposes: (i) it can be forward programmed by the overexpression of transcription factors (e.g. OLIG2 and SOX10 for ioOligodendrocyte-like cells) and (ii) it expresses the Cas9 protein in a constitutive manner to enable gene knockouts in forward programmed/differentiated cells. Thereto, three transgenes were integrated into genome safe-harbour sites (GSH) by genome engineering (see FIG. 20). Firstly, a CAG promoter-driven reverse Tet transactivator (rtTA) cassette for doxycycline-controlled transactivation of a second recombinant transgene was integrated into the ROSA26 locus. Secondly, a transcription factor (e.g. OLIG2-SOX10) under the control of doxycycline-responsive promoter was integrated into the AAVS1 locus. Lastly, the CAG promoter-driven Cas9 protein was integrated into the CLYBL locus.

### Experimental Outline

An iPSC line was cultured at all times. Editing of the safe harbour loci was performed subsequentially. All three loci were edited using CRISPR/Cas9 technology. A sgRNA which introduces a DNA double-strand break in close proximity to the chosen insertion site was selected. Cells were nucleofected with two plasmids—a plasmid expressing Cas9 and the corresponding sgRNA and a plasmid containing the insert sequence flanked by homology arms. The PAM sequence was mutated to prevent the re-cutting of Cas9. Cells harbouring the correct integration were selected (e.g. using antibiotic resistance markers with the respective antibiotic). Once selection has reached a sufficient efficiency, cells were dissociated as single cells. To obtain monoclonal lines, cells were seeded at a low density to allow outgrowth of colonies originating from a single cell. Engineered lines were submitted to two rounds of genotyping by PCR. The first round of genotyping was performed on crude lysate. Once clones with the correct integration were identified, cells were expanded, and genomic DNA was isolated for a second round of genotyping. PCRs for correct integration of 5′ and 3′ homology arms as well as the insert sequence were conducted. In addition, PCRs were performed to confirm if clones are heterozygous or homozygous for the intended integration.

### Results

The final iPSC line has been engineered to incorporate the dual safe harbour system, allowing transcription factor overexpression. It contains transcription factor(s) essential for forward programming into Oligodendrocyte-like cells. Additionally, this line constitutively expresses the Cas9 protein, facilitating further genomic manipulations.

### Example 22: Benchmarking of Cas9 Functionality in Forward Programmed Oligodendrocyte-Like Cells

### Methods

To measure the activity of Cas9 in Oligodendrocyte-like cells at D7 (after cryopreservation), a guide RNA targeting the B2 microglobulin (B2M) gene (GGCCGAGATGTCTCGCTCCG, SEQ ID NO: 2), which is expressed on all nucleated cells, was cloned into a lentiviral vector. The used vector encodes a puromycin-resistance cassette for the selection of cells with successful genomic integration. In addition, the vector contains GFP in order to estimate the multiplicity of infection. The lentiviral vector was used to transduce cells harbouring an rtTA transactivator, constitutive full-length Cas9 and the transcription factor combination necessary for forward programming under the control of a Tet-responsive promoter.

### Experimental Outline

Two clones (P1D1-heterozygous and P1F6-homozygous) were tested in this experiment. After thawing, cells were cultured using media to support differentiation into Oliogdendrocyte-like cells.

Lentiviral guide RNA delivery: Lentivirus-containing supernatants were precipitated using the PEG-it™ Virus Precipitation Solution (System Biosciences, LV810A-1), strictly adhering to the manufacturer's directions. Transduction efficacy using lentivirus was assessed on day 2 post-cell revival. The diluted lentivirus was added dropwise to the designated well or dish, ensuring even distribution by gentle agitation. Plates were then returned to the incubator for a 24-hour incubation. After this period, the medium was entirely refreshed based on the specific transduction timing. Regular cell culture maintenance was continued until day 7. Five days post guide delivery, a flow-based readout was used to assess B2M knockout efficiency. For this purpose, cells were single cell dissociated and stained with a B2M antibody. After incubation and washing steps, the signal of B2M was measured using a FACS machine. Non-transduced and unstained cells as well as cells with a non-targeting guide RNA were used as controls.

### Results

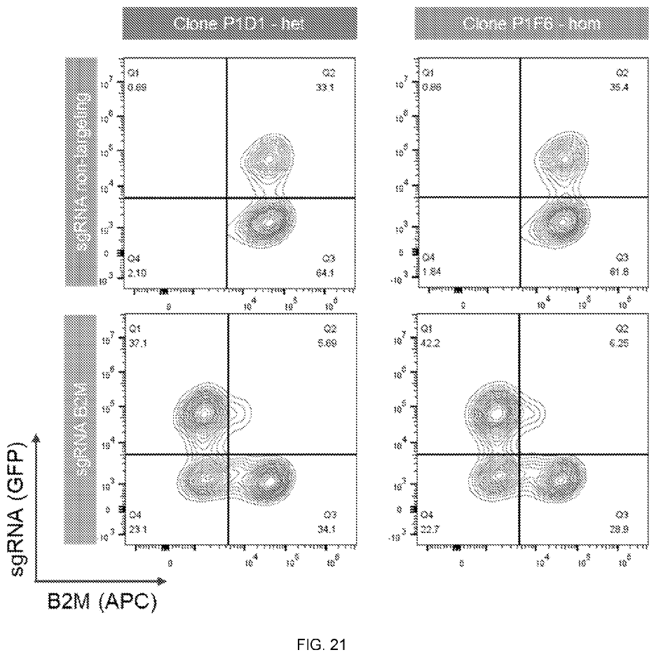

FIG. 21 shows the loss of B2M protein expression upon delivery of the specific guide RNA compared to a non-targeting control. Loss was quite pronounced and observed in the majority of the cells that received a B2M-specific guide RNA (P1D1: 84% B2M-negative cells; P1F6: 91% B2M-negative cells). In these knockout experiments, GFP serves as a surrogate marker for cells that have received a guide RNA via lentiviral delivery. Despite this, some cells exhibit successful knockout while being GFPnegative. This suggests that GFP expression may be silenced, likely due to promoter inactivity or epigenetic silencing, even though the guide RNA remains functional. Results confirm that Cas9 is active and activity is homogenous across the individual cells in the experiment.

### Example 23: Benchmarking of Cas9 Expression and Activity in Forward Programmed Glutamatergic Neurons Over Time

### Methods

Analysis of the Western blot demonstrated an apparent temporal decline in Cas9 expression across the examined time points (see Example 5). By Day 6, Cas9 expression was undetectable by Western blot analysis. However, subsequent experiments reveal that this observation was due to the sensitivity limitations of the Western blot technique. Functional assays demonstrate that Cas9 remains active even at later time points, such as Day 11, where the delivery of guide RNA results in high knockout efficiencies. These findings indicate that Cas9 expression persists over time at levels below the detection threshold of Western blot but is still sufficient for effective genome editing.

### Experimental Outline

Guide RNAs were introduced into cells using both RNAiMAX and lentivirus on Day 11 post revival. Guide RNA sequence targeting SOX11: GAGAAGATCCCGTTCATCC (SEQ ID NO: 3). Guide RNA sequence non-targeting control: GCTACCCGCGCGAGAATTGC (SEQ ID NO: 4).

Lipid-based delivery: The synthetic guide RNA delivery protocol was adapted from the manufacturer's instructions provided with the Lipofectamine® RNAiMAX Reagent (ThermoFisher, 13778075). This protocol was designed with flexibility in mind to cater to varying applications. For the transfection, microcentrifuge tubes labelled A, B, and C were set up. Tube A combined DMEM/F-12 medium with sgRNA, while Tube B had a mix of DMEM/F-12 medium and the RNAiMAX reagent. The contents of Tubes A and B were combined into Tube C, mixed well by vortexing, and incubated at room temperature for 5 minutes. This resulting sgRNA RNAIMAX mixture from Tube C was then evenly distributed in a drop-wise manner across the desired wells or dishes. Depending on the well size, the final concentrations of sgRNA when introduced to the cells were 2 μmol for 96-well plates, 10 μmol for 24-well plates, and 50 μmol for 6-well plates. Following this, plates were placed back in the incubator for 24 hours before the medium was refreshed based on the transfection timing.

Lentiviral delivery: Lentivirus-containing supernatants were precipitated using the PEG-it™ Virus Precipitation Solution (System Biosciences, LV810A-1), strictly adhering to the manufacturer's directions. For the transduction, diluted lentivirus was added drop-wise to the designated well or dish, ensuring even distribution by gentle agitation. Plates were then returned to the incubator for a 24-hour incubation. After this period, the medium was entirely refreshed based on the specific transduction timing. Regular cell culture maintenance was then continued, and the selected readout method was applied at the appropriate time.

Five days post guide delivery, cells were used for immunofluorescence staining. To avoid cell detachment, they were fixed by exchanging half of the media volume with 8% Paraformaldehyde (PFA) from ThermoFisher (Cat. No. 11586711), followed by a second fixation step with 4% PFA only. After multiple PBS (Gibco, 20012027) washes, cells were incubated with primary antibody (SOX11 Monoclonal Antibody from ThermoFisher, 14-9773-82) overnight at 4° C. Post-wash, secondary antibodies and DAPI (Biotechne, 5748/10) were incubated for an hour. Following a series of washes with 0.1% Triton-X (Sigma, T8787), cells were stored in PBS until their subsequent analysis. To assess the knock-out efficiency, an advanced image analysis pipeline was employed. This pipeline incorporated Deep Learning methodologies, specifically tailored for precise recognition of cell nuclei. Utilizing this approach, the intensity and distribution of the DAPI signal, indicative of cell nuclei, as well as the SOX11 signal, were meticulously quantified. The derived data, based on the correlation between DAPI and SOX11 signals, provided an accurate measure of the knock-out efficiency of SOX11 at the protein level.

### Results

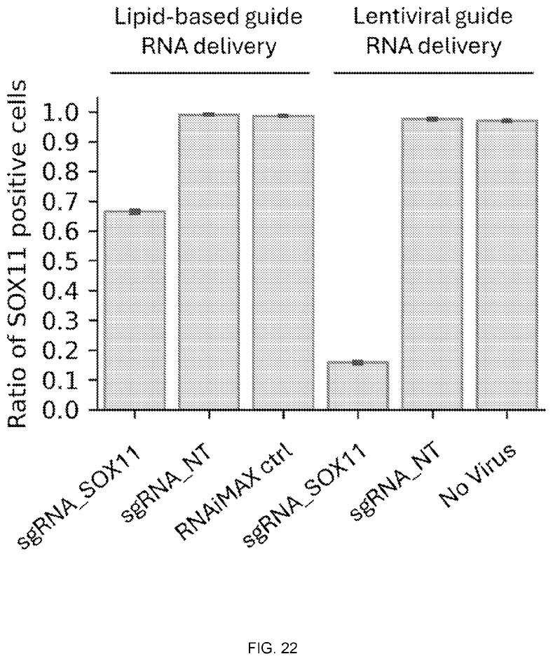

Guide RNA was delivered to cells using lipid-based and lentiviral guide delivery methods on Day 11 post-revival. On Day 16, SOX11 expression was examined with immunofluorescence staining. DAPI staining marked the cell nuclei, and a non-targeting guide was used as a control. Image analysis indicates a significant knock-out of SOX11 protein following introduction of the SOX11-specific gRNA (see FIG. 22).

1. A method of modulating the expression of an endogenous gene in a somatic cell derived from an induced pluripotent stem cell (iPSC), comprising the steps of:
(i) providing an engineered iPSC capable of stable or inducible expression of a CRISPR enzyme, a catalytically-inactive CRISPR enzyme or a derivative thereof;
(ii) inducing the engineered iPSC to differentiate into the somatic cell; and
(iii) introducing a guide RNA (gRNA) sequence into the somatic cell obtained in step (ii), wherein said gRNA sequence is complementary to the endogenous gene,
thereby modulating the expression of the endogenous gene in the somatic cell following differentiation.

2. The method of claim 1, wherein the CRISPR enzyme is Cas9 or the catalytically-inactive CRISPR enzyme is catalytically-inactive Cas9 (dCas9).

3. The method of claim 1, wherein the engineered iPSC comprises an inserted genetic sequence encoding the CRISPR enzyme, catalytically-inactive CRISPR enzyme or a derivative thereof operably linked to a promoter.

4. (canceled)

5. The method of claim 1, wherein the engineered iPSC comprises:
(a) an inserted genetic sequence encoding a transcriptional regulator protein at a first genomic safe harbour (GSH) site;
(b) an inserted inducible cassette comprising a genetic sequence encoding a transcription factor operably linked to an inducible promoter at a second GSH site, wherein said inducible promoter is regulated by the transcriptional regulator protein; and
(c) a genetic sequence encoding the CRISPR enzyme, catalytically-inactive CRISPR enzyme or derivative thereof at a third GSH site,
wherein the first, second, and third GSH sites are different.

6-8. (canceled)

9. The method of claim 5, wherein the first, second, and/or third GSH sites are selected from the group consisting of: the hROSA26 locus, the AAVS1 locus, the CLYBL gene and the CCR5 gene.

10-11. (canceled)

12. The method of claim 1, wherein step (ii) comprises forward programming by expression of one or more lineage-specific transcription factors.

13. (canceled)

14. The method of claim 5, wherein transcription of the genetic sequence encoding the transcription factor in the second GSH site results in forward programming of the engineered iPSC into the somatic cell.

15. The method of claim 1, wherein step (ii) comprises directed differentiation by culturing the iPSC under conditions required to induce differentiation into the somatic cell.

16-17. (canceled)

18. The method of claim 1, wherein the gRNA is introduced in step (iii) within 6 days of differentiation, or within 1-3 days from differentiation.

19. The method of claim 1, wherein the endogenous gene contains an insertion or deletion after introduction of the gRNA sequence, thus disrupting the coding sequence of the endogenous gene.

20. (canceled)

21. The method of claim 1, wherein the expression of the endogenous gene is activated or repressed.

22-26. (canceled)

27. The method of claim 1, wherein the gRNA sequence is complementary to a coding exon of the endogenous gene or a transcription start site (TSS) for the endogenous gene.

28. The method of claim 1, wherein two or more gRNA sequences are introduced.

29. The method of claim 28, wherein each of the gRNA sequences are complementary to alternative or different endogenous genes, or are complementary to more than one alternative sequences of a TSS for the endogenous gene.

30. The method of claim 1, wherein the engineered iPSC is frozen and subsequently thawed prior to differentiation.

31. The method of claim 1, wherein differentiation is induced and the differentiated cells are frozen and subsequently thawed.

32. The method of claim 1, wherein the engineered iPSC is a human, marsupial, non-human primate, camelid or livestock animal cell.

33. The method of claim 1, wherein the somatic cell is a neuronal cell or a glial cell.

34. A cell with a modified genome that comprises:
(i) an inserted genetic sequence encoding a transcriptional regulator protein at a first GSH site;
(ii) an inserted inducible cassette comprising a genetic sequence operably linked to an inducible promoter at a second GSH site, wherein said inducible promoter is regulated by the transcriptional regulator protein; and
(iii) an inserted genetic sequence encoding a CRISPR enzyme, a catalytically-inactive CRISPR enzyme or a derivative thereof operably linked to a promoter;
wherein the first and second GSH sites are different, and
wherein the genetic sequence encoding the CRISPR enzyme, catalytically-inactive CRISPR enzyme or derivative thereof is inserted at a site which is different to the first and second GSH sites.

35-41. (canceled)

42. A cell obtained by the method of claim 1.

43-47. (canceled)
